SEQUENCE LISTING (37 sequences)

SEQ ID NO: 1 - RNA, 29 nt, synthetic construct
  source 1..29: mol_type = other RNA
  misc_binding 1: bound_moiety = triethylene glycol spacer
  misc_binding 29: bound_moiety = Cholesterol-TEG
  modified_base 1: mod_base = cm
  modified_base 2: mod_base = OTHER; note = locked nucleic acid base
  modified_base 3: mod_base = OTHER; note = 2'-O-methylated base
  modified_base 4: mod_base = OTHER; note = locked nucleic acid base
  modified_base 5: mod_base = gm
  modified_base 6: mod_base = OTHER; note = 2'-O-methylated base
  modified_base 7: mod_base = OTHER; note = locked nucleic acid base
  modified_base 8: mod_base = cm
  modified_base 9: mod_base = gm
  modified_base 10: mod_base = OTHER; note = locked nucleic acid base
  modified_base 11: mod_base = OTHER; note = 2'-O-methyladenine
  modified_base 12: mod_base = OTHER; note = locked nucleic acid base
  modified_base 13: mod_base = OTHER; note = 2'-O-methyladenine
  modified_base 14: mod_base = OTHER; note = 2'-O-methyluracil
  modified_base 15: mod_base = OTHER; note = locked nucleic acid base
  modified_base 16..17: mod_base = cm
  modified_base 18: mod_base = OTHER; note = locked nucleic acid base
  modified_base 19..20: mod_base = gm
  modified_base 21: mod_base = OTHER; note = locked nucleic acid base
  modified_base 22..23: mod_base = OTHER; note = 2'-O-methyladenine
  modified_base 24: mod_base = OTHER; note = locked nucleic acid thymine
  modified_base 25: mod_base = gm
  modified_base 26: mod_base = OTHER; note = 2'-O-methyuracil
  modified_base 27..29: mod_base = OTHER; note = locked nucleic acid base
  modified_base 1..3: mod_base = OTHER; note = phosphorothioate backbone
  modified_base 22..29: mod_base = OTHER; note = phosphorothioate backbone
cgaagaacggaaatccctggcaatgtgat

SEQ ID NO: 2 - RNA, 21 nt, synthetic construct
  source 1..21: mol_type = other RNA
  modified_base 1..2: mod_base = OTHER; note = locked nucleic acid thymine
  modified_base 3: mod_base = OTHER; note = 2'-O-methyladenosine
  modified_base 4: mod_base = OTHER; note = locked nucleic acid thymine
  modified_base 5..8: mod_base = OTHER; note = 2'-O-methylated base
  modified_base 9: mod_base = OTHER; note = 2'-fluoro G
  modified_base 10: mod_base = OTHER; note = 2'-O methyl U
  modified_base 11..13: mod_base = OTHER; note = 2'-fluoro A
  modified_base 14..18: mod_base = OTHER; note = 2'-O methylated base
  modified_base 19: mod_base = OTHER; note = locked nucleic acid T
  modified_base 20..21: mod_base = OTHER; note = 2'-O-methyladenosine
  modified_base 1..3: mod_base = OTHER; note = phosphorothioate backbone
  modified_base 19..21: mod_base = OTHER; note = phosphorothioate backbone
ttatatcagtaaagagattaa

SEQ ID NO: 3 - RNA, 11 nt, synthetic construct
  source 1..11: mol_type = other RNA
  modified_base 1..11: mod_base = OTHER; note = 2'-O methylated base
  misc_feature 11: note = The 3' terminus is attached to a C3 spacer that is joined to SEQ ID NO: 4.
tccgttcttcg

SEQ ID NO: 4 - RNA, 21 nt, synthetic construct
  source 1..21: mol_type = other RNA
  modified_base 1: mod_base = OTHER; note = 2'-O methyl U
  modified_base 2: mod_base = OTHER; note = 2'-fluoro U
  modified_base 3..5: mod_base = OTHER; note = 2'-O methylated base
  modified_base 6: mod_base = OTHER; note = locked nucleic acid C
  modified_base 7..11: mod_base = OTHER; note = 2'-O methylated base
  modified_base 12: mod_base = OTHER; note = 2'-fluoro A
  modified_base 13: mod_base = cm
  modified_base 14: mod_base = OTHER; note = 2'-fluoro U
  modified_base 15..21: mod_base = OTHER; note = 2'-O methylated base
  modified_base 1..3: mod_base = OTHER; note = phosphorothioate backbone
  misc_feature 21: note = The 3' terminus is attached to a C3 spacer that is joined to SEQ ID NO: 5.
ttaatctctttactgatataa

SEQ ID NO: 5 - RNA, 11 nt, synthetic construct
  source 1..11: mol_type = other RNA
  modified_base 1..11: mod_base = OTHER; note = 2'-O-methylated base
  modified_base 1^2: mod_base = OTHER; note = phosphorothioate bond
tgccagggatt

SEQ ID NO: 6 - RNA, 11 nt, synthetic construct
  source 1..11: mol_type = other RNA
  modified_base 1..11: mod_base = OTHER; note = 2'-O methylated base
  misc_feature 11: note = The 3' terminus is attached to a C3 spacer that is joined to SEQ ID NO: 7.
tccgttcttcg

SEQ ID NO: 7 - RNA, 33 nt, synthetic construct
  source 1..33: mol_type = other RNA
  modified_base 1: mod_base = OTHER; note = 2'-O methyl U
  modified_base 2: mod_base = OTHER; note = 2'-fluoro U
  modified_base 3..5: mod_base = OTHER; note = 2'-O methylated base
  modified_base 6: mod_base = OTHER; note = 2'-fluoro C
  modified_base 7..11: mod_base = OTHER; note = 2'-O methylated base
  modified_base 12: mod_base = OTHER; note = 2'-fluoro A
  modified_base 13: mod_base = cm
  modified_base 14: mod_base = OTHER; note = 2'-fluoro U
  modified_base 15..33: mod_base = OTHER; note = 2'-O methylated base
ttaatctctttactgatataattgccagggatt

SEQ ID NO: 8 - RNA, 33 nt, synthetic construct
  source 1..33: mol_type = other RNA
  modified_base 1..13: mod_base = OTHER; note = 2'-O methylated base
  modified_base 13..15: mod_base = OTHER; note = phosphorothioate backbone
  modified_base 14: mod_base = OTHER; note = 2'-fluoro U
  modified_base 15..17: mod_base = OTHER; note = 2'-O-methylated base
  modified_base 18: mod_base = OTHER; note = 2'-fluoro C
  modified_base 19..23: mod_base = OTHER; note = 2'-O methylated base
  modified_base 24: mod_base = OTHER; note = 2'-fluoro A
  modified_base 25: mod_base = cm
  modified_base 26: mod_base = OTHER; note = 2'-fluoro U
  modified_base 27..33: mod_base = OTHER; note = 2'-O methylated base
  misc_feature 33: note = The 3' terminus is attached to a C3 spacer that is joined to SEQ ID NO: 9.
tccgttcttcgattaatctctttactgatataa

SEQ ID NO: 9 - RNA, 11 nt, synthetic construct
  source 1..11: mol_type = other RNA
  modified_base 1..11: mod_base = OTHER; note = 2'-O methylated base
  modified_base 1^2: mod_base = OTHER; note = phosphorothioate bond
tgccagggatt

SEQ ID NO: 10 - RNA, 45 nt, synthetic construct
  source 1..45: mol_type = other RNA
  modified_base 1..13: mod_base = OTHER; note = 2'-O methylated base
  modified_base 14: mod_base = OTHER; note = 2'-fluoro U
  modified_base 15..17: mod_base = OTHER; note = 2'-O methylated base
  modified_base 18: mod_base = OTHER; note = 2'-fluoro C
  modified_base 19..23: mod_base = OTHER; note = 2'-O-methylated base
  modified_base 24: mod_base = OTHER; note = 2'-fluoro A
  modified_base 25: mod_base = cm
  modified_base 26: mod_base = OTHER; note = 2'-fluoro U
  modified_base 27..45: mod_base = OTHER; note = 2'-O methylated base
  modified_base 13..15: mod_base = OTHER; note = phosphorothioate backbone
  modified_base 33..35: mod_base = OTHER; note = phosphorothioate backbone
tccgttcttcgattaatctctttactgatataattgccagggatt

SEQ ID NO: 11 - RNA, 44 nt, synthetic construct
  source 1..44: mol_type = other RNA
  modified_base 1..12: mod_base = OTHER; note = 2'-O methyl base
  modified_base 13: mod_base = OTHER; note = 2'-fluoro U
  modified_base 14..16: mod_base = OTHER; note = 2'-O-methyl base
  modified_base 17: mod_base = OTHER; note = 2'-fluoro C
  modified_base 18..22: mod_base = OTHER; note = 2'-O-methyl base
  modified_base 23: mod_base = OTHER; note = 2'-fluoro A
  modified_base 24: mod_base = cm
  modified_base 25: mod_base = OTHER; note = 2'-fluoro U
  modified_base 26..44: mod_base = OTHER; note = 2'-O-methyl base
  modified_base 32..34: mod_base = OTHER; note = phosphorothioate backbone
  modified_base 12..14: mod_base = OTHER; note = phosphorothioate backbone
tccgttcttcgttaatctctttactgatataattgccagggatt

SEQ ID NO: 12 - RNA, 43 nt, synthetic construct
  source 1..43: mol_type = other RNA
  modified_base 1..12: mod_base = OTHER; note = 2'-O-methyl base
  modified_base 13: mod_base = OTHER; note = 2'-fluoro U
  modified_base 14..16: mod_base = OTHER; note = 2'-O-methyl base
  modified_base 17: mod_base = OTHER; note = 2'-fluoro C
  modified_base 18..22: mod_base = OTHER; note = 2'-O-methyl base
  modified_base 23: mod_base = OTHER; note = 2'-fluoro A
  modified_base 24: mod_base = cm
  modified_base 25: mod_base = OTHER; note = 2'-fluoro U
  modified_base 26..43: mod_base = OTHER; note = 2'-O-methyl base
  modified_base 12..14: mod_base = OTHER; note = phosphorothioate backbone
  modified_base 32..34: mod_base = OTHER; note = phosphorothioate backbone
tccgttcttcgttaatctctttactgatataatgccagggatt

SEQ ID NO: 13 - RNA, 21 nt, synthetic construct
  source 1..21: mol_type = other RNA
  misc_binding 1: bound_moiety = Cy3
  modified_base 1: mod_base = OTHER; note = 2'-O-methyl U
  modified_base 2: mod_base = OTHER; note = locked nucleic acid A
  modified_base 3: mod_base = cm
  modified_base 4: mod_base = OTHER; note = 2'-O-methyladenosine
  modified_base 5..6: mod_base = gm
  modified_base 7: mod_base = OTHER; note = 2'-fluoro A
  modified_base 8: mod_base = OTHER; note = 2'-O-methyladenosine
  modified_base 9..11: mod_base = OTHER; note = 2'-fluoro base
  modified_base 12..21: mod_base = OTHER; note = 2'-O-methyl base
  modified_base 1..3: mod_base = OTHER; note = phosphorothioate backbone
  modified_base 19..21: mod_base = OTHER; note = phosphorothioate backbone
tacaggaaaagccaaacaaca

SEQ ID NO: 14 - RNA, 31 nt, synthetic construct
  source 1..31: mol_type = other RNA
  misc_binding 1: bound_moiety = triethylene glycol spacer
  misc_binding 31: bound_moiety = Cholesterol-TEG
  modified_base 1: mod_base = cm
  modified_base 2: mod_base = OTHER; note = locked nucleic acid T
  modified_base 3: mod_base = OTHER; note = 2'-O-methyl U
  modified_base 4: mod_base = cm
  modified_base 5: mod_base = OTHER; note = locked nucleic acid A
  modified_base 6..7: mod_base = cm
  modified_base 8: mod_base = OTHER; note = locked nucleic acid A
  modified_base 9: mod_base = cm
  modified_base 10: mod_base = OTHER; note = locked nucleic acid
  modified_base 11..13: mod_base = OTHER; note = 2'-O-methyl base
  modified_base 14: mod_base = OTHER; note = locked nucleic acid base
  modified_base 15..16: mod_base = OTHER; note = 2'-O-methyl base
  modified_base 17: mod_base = OTHER; note = locked nucleic acid T
  modified_base 18: mod_base = gm
  modified_base 19: mod_base = OTHER; note = locked nucleic acid base
  modified_base 20: mod_base = cm
  modified_base 21: mod_base = OTHER; note = 2'-O-methyladenosine
  modified_base 22: mod_base = OTHER; note = locked nucleic acid base
  modified_base 23..25: mod_base = OTHER; note = 2'-O-methyl base
  modified_base 26: mod_base = OTHER; note = locked nucleic acid base
  modified_base 27: mod_base = OTHER; note = 2'-O-methyladenosine
  modified_base 28: mod_base = OTHER; note = locked nucleic acid base
  modified_base 29: mod_base = cm
  modified_base 30: mod_base = OTHER; note = locked nucleic acid base
  modified_base 31: mod_base = OTHER; note = 2'-O-methyladenosine
  modified_base 23..31: mod_base = OTHER; note = phosphorothioate backbone
cttcaccacctctcagtggcaatgcgaccaa

SEQ ID NO: 15 - RNA, 33 nt, synthetic construct
  source 1..33: mol_type = other RNA
  modified_base 1..13: mod_base = OTHER; note = 2'-O-methyl base
  modified_base 14: mod_base = OTHER; note = 2'-fluoro G
  modified_base 15..17: mod_base = OTHER; note = 2'-O-methyl base
  modified_base 18: mod_base = OTHER; note = 2'-fluoro U
  modified_base 19..23: mod_base = OTHER; note = 2'-O-methyl base
  modified_base 24: mod_base = OTHER; note = 2'-fluoro U
  modified_base 25: mod_base = OTHER; note = 2'-O-methyl base
  modified_base 26: mod_base = OTHER; note = 2'-fluoro U
  modified_base 27..33: mod_base = OTHER; note = 2'-O-methyl base
  misc_feature 33: note = The 3' terminus is attached to a C3 space via a phosphorothioate backbone, which is joined to SEQ ID NO: 16.
  modified_base 13..15: mod_base = OTHER; note = phosphorothioate backbone
aggtggtgaagatgttgtttggcttttcctgta

SEQ ID NO: 16 - RNA, 11 nt, synthetic construct
  source 1..11: mol_type = other RNA
  modified_base 1..11: mod_base = OTHER; note = 2'-O-methyl base
  modified_base 1^2: mod_base = OTHER; note = phosphorothioate backbone
ttgccactgag

SEQ ID NO: 17 - RNA, 25 nt, synthetic construct
  source 1..25: mol_type = other RNA
  misc_binding 1: bound_moiety = C3 spacer
  modified_base 1..2: mod_base = OTHER; note = locked nucleic acid
  modified_base 11: mod_base = OTHER; note = 2'-O methyl base
  modified_base 13..14: mod_base = gm
  modified_base 15: mod_base = cm
  modified_base 17: mod_base = OTHER; note = 2'-O-methyl base
  modified_base 21..25: mod_base = OTHER; note = 2'-O-methyl base
  modified_base 1^2: mod_base = OTHER; note = phosphorothioate bond
  modified_base 23..25: mod_base = OTHER; note = phosphorothioate backbone
cgagtgttgtttggcttttcctgtt

SEQ ID NO: 18 - RNA, 11 nt, synthetic construct
  source 1..11: mol_type = other RNA
  modified_base 1..11: mod_base = OTHER; note = 2'-O-methyl base
  misc_feature 11: note = The 3' terminus is attached to a C3 spacer, which is joined to SEQ ID NO: 19.
aggtggtgaag

SEQ ID NO: 19 - RNA, 23 nt, synthetic construct
  source 1..23: mol_type = other RNA
  misc_feature 23: note = The 3' terminus is attached to a C3 spacer, which is joined to SEQ ID NO: 20
  modified_base 1^2: mod_base = OTHER; note = phosphorothioate backbone
  modified_base 22^23: mod_base = OTHER; note = phosphorothioate bond
  modified_base 1: mod_base = cm
  modified_base 2: mod_base = OTHER; note = locked nucleic acid base
  modified_base 3: mod_base = gm
  modified_base 23: mod_base = gm
caggaaaagccaaacaacactcg

SEQ ID NO: 20 - RNA, 12 nt, synthetic construct
  source 1..12: mol_type = other RNA
  modified_base 1..12: mod_base = OTHER; note = 2'-O-methyl base
attgccattgag

SEQ ID NO: 21 - RNA, 23 nt, synthetic construct
  source 1..23: mol_type = other RNA
  modified_base 15..23: mod_base = OTHER; note = 2'-O-methyl base
  modified_base 14: mod_base = OTHER; note = 2'-fluoro U
  modified_base 13: mod_base = cm
  modified_base 12: mod_base = OTHER; note = 2'-fluoro A
  modified_base 7..11: mod_base = OTHER; note = 2'-O-methyl base
  modified_base 6: mod_base = OTHER; note = 2'-fluoro C
  modified_base 3..5: mod_base = OTHER; note = 2'-O-methyl base
  modified_base 2: mod_base = OTHER; note = 2'-fluoro base
  modified_base 1: mod_base = OTHER; note = 2'-O-methyl base
  modified_base 1..3: mod_base = OTHER; note = phosphorothioate backbone
  modified_base 21..23: mod_base = OTHER; note = phosphorothioate backbone
ttaatatagtcatttctctaatt

SEQ ID NO: 22 - RNA, 21 nt, synthetic construct
  source 1..21: mol_type = other RNA
  modified_base 1..3: mod_base = OTHER; note = phosphorothioate backbone
  modified_base 19..21: mod_base = OTHER; note = phosphorothioate backbone
  modified_base 1..3: mod_base = OTHER; note = 2'-O-methyl base
  modified_base 4: mod_base = OTHER; note = locked nucleic acid T
  modified_base 5..8: mod_base = OTHER; note = 2'-O-methyl base
  modified_base 9: mod_base = OTHER; note = 2'-fluoro G
  modified_base 10: mod_base = OTHER; note = 2'-O-methyl U
  modified_base 11..13: mod_base = OTHER; note = 2'-fluoro A
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 23 - RNA, 20 nt, synthetic construct
  source 1..20: mol_type = other RNA
  modified_base 1..3: mod_base = OTHER; note = phosphorothioate backbone
  modified_base 13..20: mod_base = OTHER; note = phosphorothioate backbone
  modified_base 1: mod_base = OTHER; note = 2'-O-methyl U
  modified_base 2: mod_base = OTHER; note = 2'-fluoro U
  modified_base 3: mod_base = OTHER; note = 2'-O-methyladenosine
  modified_base 4: mod_base = OTHER; note = 2-fluoro A
  modified_base 5: mod_base = OTHER; note = 2'-O-methyl U
  modified_base 6: mod_base = OTHER; note = 2'-fluoro base
  modified_base 7: mod_base = OTHER; note = 2'-O-methyl base
  modified_base 8: mod_base = OTHER; note = 2'-fluoro base
  modified_base 9: mod_base = OTHER; note = 2'-O-methyl base
  modified_base 10: mod_base = OTHER; note = 2'-fluoro base
  modified_base 11: mod_base = OTHER; note = 2'-O-methyl base
  modified_base 12: mod_base = OTHER; note = 2'-fluoro base
  modified_base 13: mod_base = cm
  modified_base 14: mod_base = OTHER; note = 2'-fluoro base
  modified_base 15: mod_base = gm
  modified_base 16: mod_base = OTHER; note = 2'-fluoro base
  modified_base 17: mod_base = OTHER; note = 2'-O-methyl base
  modified_base 18: mod_base = OTHER; note = 2'-fluoro base
  modified_base 19: mod_base = OTHER; note = 2'-O-methyl base
  modified_base 20: mod_base = OTHER; note = 2'-fluoro base
ttaatctctttactgatata

SEQ ID NO: 24 - RNA, 15 nt, synthetic construct
  source 1..15: mol_type = other RNA
  misc_binding 1: bound_moiety = C3 spacer
  misc_binding 15: bound_moiety = Cholesterol-TEG
  modified_base 1..3: mod_base = OTHER; note = phosphorothioate backbone
  modified_base 13..15: mod_base = OTHER; note = phosphorothioate backbone
  modified_base 1: mod_base = OTHER; note = 2'-fluoro base
  modified_base 2: mod_base = OTHER; note = 2'-O-methyladenosine
  modified_base 3: mod_base = OTHER; note = 2'-fluoro base
  modified_base 4: mod_base = OTHER; note = 2'-O-methyl base
  modified_base 5: mod_base = OTHER; note = 2'-fluoro base
  modified_base 6: mod_base = OTHER; note = 2'-O-methyladenosine
  modified_base 7: mod_base = OTHER; note = 2'-fluoro base
  modified_base 8: mod_base = gm
  modified_base 9: mod_base = OTHER; note = 2'-fluoro base
  modified_base 10: mod_base = gm
  modified_base 11: mod_base = OTHER; note = 2'-fluoro base
  modified_base 12: mod_base = OTHER; note = 2'-O-methyl base
  modified_base 13: mod_base = OTHER; note = 2'-fluoro base
  modified_base 14: mod_base = OTHER; note = 2'-O-methyladenosine
  modified_base 15: mod_base = OTHER; note = 2'-fluoro base
cagtaaagagattaa

SEQ ID NO: 25 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  misc_binding 1: bound_moiety = Cy3 dye
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 26 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  misc_binding 1: bound_moiety = 5' spacer 9
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 27 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  misc_binding 21: bound_moiety = 3' inverted dT
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 28 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  misc_binding 21: bound_moiety = 3' Cy3Sp
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 29 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  misc_binding 21: bound_moiety = 3' spacer 9
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 30 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  misc_binding 1: bound_moiety = Cy3 dye
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
  misc_binding 21: bound_moiety = 3' inverted dT
ttatatcagtaaagagattaa

SEQ ID NO: 31 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = 2-O-methyl base
  modified_base 11..13: mod_base = OTHER; note = 2'-fluoro base
  modified_base 15..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
  modified_base 14: mod_base = OTHER; note = locked nucleic acid base
ttatatcagtaaagagattaa

SEQ ID NO: 32 - RNA, 21 nt, synthetic construct
  source 1..21: mol_type = other RNA
  modified_base 1^2: mod_base = OTHER; note = phosphorothioate bond
  modified_base 19..21: mod_base = OTHER; note = phosphorothioate backbone
  modified_base 1..2: mod_base = OTHER; note = locked nucleic acid T
  modified_base 3: mod_base = OTHER; note = 2'-O-methyladenosine
  modified_base 4: mod_base = OTHER; note = locked nucleic acid T
  modified_base 5..8: mod_base = OTHER; note = 2'-O-methyl base
  modified_base 9: mod_base = OTHER; note = 2-fluoro base
  modified_base 10: mod_base = OTHER; note = locked nucleic acid base T
  modified_base 11..13: mod_base = OTHER; note = 2'-fluoro base
  modified_base 14..18: mod_base = OTHER; note = 2'-O-methyl base
  modified_base 19: mod_base = OTHER; note = locked nucleic acid T
  modified_base 20..21: mod_base = OTHER; note = 2'-O-methyladenosine
ttatatcagtaaagagattaa

SEQ ID NO: 33 - RNA, 24 nt, synthetic construct
  source 1..24: mol_type = other RNA
  misc_feature 1: note = The 5 terminus is attached to a spacer 9 via phosphorothioate backbone.
  misc_binding 24: bound_moiety = 3' AmMC6
  modified_base 1: mod_base = gm
  modified_base 2: mod_base = OTHER; note = locked nucleic acid base
  modified_base 3..4: mod_base = cm
  modified_base 5: mod_base = OTHER; note = locked nucleic acid base
  modified_base 6..8: mod_base = OTHER; note = 2'-O-methyl base
  modified_base 9: mod_base = OTHER; note = locked nucleic acid base T
  modified_base 10: mod_base = gm
  modified_base 11: mod_base = cm
  modified_base 12: mod_base = gm
  modified_base 13: mod_base = OTHER; note = locked nucleic acid base T
  modified_base 14..18: mod_base = OTHER; note = 2'-O-methyl base
  modified_base 19: mod_base = OTHER; note = locked nucleic acid base
  modified_base 20..21: mod_base = cm
  modified_base 22..23: mod_base = OTHER; note = locked nucleic acid base
  modified_base 1..3: mod_base = OTHER; note = phosphorothioate backbone
  modified_base 12..24: mod_base = OTHER; note = phosphorothioate backbone
gaccaagctgcgtgacacaccact

SEQ ID NO: 34 - RNA, 37 nt, synthetic construct
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  modified_base 1..3: mod_base = OTHER; note = phosphorothioate backbone
  modified_base 35..37: mod_base = OTHER; note = phosphorothioate backbone
  modified_base 15..17: mod_base = OTHER; note = phosphorothioate backbone
  modified_base 1: mod_base = cm
  modified_base 2..15: mod_base = OTHER; note = 2'-O-methyl base
cacgcagcttggtctgttgtttggcttttcctgtaca

SEQ ID NO: 35 - RNA, 24 nt, synthetic construct
  source 1..24: mol_type = other RNA
  misc_binding 24: bound_moiety = 3' AmMC6
  modified_base 1: mod_base = gm
  modified_base 2: mod_base = OTHER; note = locked nucleic acid base
  modified_base 3..4: mod_base = cm
  modified_base 5: mod_base = OTHER; note = locked nucleic acid base
  modified_base 6..8: mod_base = OTHER; note = 2'-O-methyl base
  modified_base 9: mod_base = OTHER; note = locked nucleic acid base T
  modified_base 10: mod_base = gm
  modified_base 11: mod_base = cm
  modified_base 12: mod_base = gm
  modified_base 13: mod_base = OTHER; note = locked nucleic acid base T
  modified_base 14..18: mod_base = OTHER; note = 2'-O-methyl base
  modified_base 19: mod_base = OTHER; note = locked nucleic acid base
  modified_base 20..21: mod_base = cm
  modified_base 22..23: mod_base = OTHER; note = locked nucleic acid base
  modified_base 1..3: mod_base = OTHER; note = phosphorothioate backbone
  modified_base 12..24: mod_base = OTHER; note = phosphorothioate backbone
gaccaagctgcgtgacacaccact

SEQ ID NO: 36 - RNA, 37 nt, synthetic construct; identical to SEQ ID NO: 34
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  modified_base 35..37: mod_base = OTHER; note = phosphorothioate backbone
  modified_base 15..17: mod_base = OTHER; note = phosphorothioate backbone
  modified_base 1: mod_base = cm
  modified_base 2..15: mod_base = OTHER; note = 2'-O-methyl base

SEQ ID NO: 37 - RNA, 37 nt, synthetic construct; identical to SEQ ID NO: 34
  source 1..37: mol_type = other RNA
  modified_base 16: mod_base = OTHER; note = 2'-fluoro base
  modified_base 17..19: mod_base = OTHER; note = 2'-O-methyl base
  modified_base 20: mod_base = OTHER; note = 2'-fluoro base
  modified_base 21..27: mod_base = OTHER; note = 2'-O-methyl base
  modified_base 28: mod_base = OTHER; note = 2'-fluoro base
  modified_base 29: mod_base = OTHER; note = 2'-O-methyl base
  modified_base 30: mod_base = OTHER; note = 2'-fluoro base
  modified_base 31..37: mod_base = OTHER; note = 2'-O-methyl base
  modified_base 35..37: mod_base = OTHER; note = phosphorothioate backbone
  modified_base 15..17: mod_base = OTHER; note = phosphorothioate backbone
  modified_base 1: mod_base = cm
  modified_base 2..15: mod_base = OTHER; note = 2'-O-methyl base
  misc_feature 1: note = 5' spacer 9

(19) United States
(12) Patent Application Publication
(10) Pub. No.: US 2025/0188454 A1
(43) Pub. Date: Jun. 12, 2025

(54) SIGNAL ACTIVATABLE NUCLEIC ACID CONSTRUCTS WITH WOBBLE BASE PAIRINGS

(71) Applicant: Switch Therapeutics Inc., South San Francisco, CA (US)

(72) Inventors: Si-ping Han, South San Francisco, CA (US); Lisa Scherer, South San Francisco, CA (US); Robert Duff, South San Francisco, CA (US)

(21) Appl. No.: 18/575,481

(22) PCT Filed: Jul. 5, 2022
(86) PCT No.: PCT/US2022/073428

§ 371 Date: Dec. 29, 2023

Related U.S. Application Data
(60) Provisional application No. 63/218,850, filed on Jul. 6, 2021.

(51) Int. Cl. C12N 15/113

(52) U.S. Cl. CPC C12N 15/113; C12N 2310/14; C12N 2310/15; C12N 2310/315; C12N 2310/3231; C12N 2310/331; C12N 2310/3519; C12N 2310/533; C12N 2320/52

(57) ABSTRACT

Provided herein include conditionally activatable small interfering RNA (siRNA) complexes, components, compositions, and related methods and systems. The siRNA complex can be conditionally activated upon a complementary binding to an input nucleic acid strand (e.g., a biomarker gene specific to disease-related cells) through a sequence in a sensor nucleic acid strand of the nucleic acid complex. The activated nucleic acid complex can release a potent RNAi duplex formed by a core nucleic acid strand and a passenger nucleic acid strand, which can specifically inhibit a target RNA.

### CROSS-REFERENCE TO RELATED APPLICATIONS

This application claims the benefit under 35 U.S.C. § 119(e) to U.S. Provisional Patent Application No. 63/218,850 filed on Jul. 6, 2021, the content of which is incorporated herein by reference in its entirety for all purposes.

### REFERENCE TO SEQUENCE LISTING

The present application is being filed along with a Sequence Listing in electronic format. The Sequence Listing is provided as a file entitled 75EN-329792-WO, created Jul. 4, 2022, which is 238 kilobytes in size. The information in the electronic format of the Sequence Listing is incorporated herein by reference in its entirety.

### BACKGROUND

### Field

The present disclosure relates generally to the field of nucleic acid, for example, conditionally activatable small interfering RNA complexes.

### Description of the Related Art

Despite emerging developments in the field of dynamic nuclei acid nanotechnology and biomolecular computing, there is still a challenge to develop targeted RNAi therapy that can use nuclei acid logic switches to sense RNA transcripts (such as mRNAs and miRNAs), thereby restricting RNA interfering (RNAi) therapy to specific populations of disease-related cells. In particular, there is a need to develop targeted and conditionally activated RNAi therapy with improved drug potency, sensitivity, and stability, low design complexity, and low dosage requirement.

### SUMMARY

Disclosed herein include signal activatable nucleic acid complexes with wobble base pairings. The nucleic acid complex can, in some embodiments, comprise: a first nucleic acid strand comprising 20-70 linked nucleosides; a second nucleic acid strand binding to a central region of the first nucleic acid strand to form a first nucleic acid duplex; and a third nucleic acid strand binding to a 5′ region and a 3′ region of the first nucleic acid strand to form a second nucleic acid duplex, where the third nucleic acid strand comprises an overhang, the overhang is not complementary to the first nucleic acid strand and is capable of binding to an input nucleic acid strand to cause the displacement of the third nucleic acid strand from the first nucleic acid strand, and the second nucleic acid duplex comprises at least one wobble base pair.

The at least one wobble base pair can be, for example, a guanine-uracil (G-U) wobble base pair, a hypoxanthine-uracil (I-U) wobble base pair, a hypoxanthine-adenine (I-A) wobble base pair, a hypoxanthine-cytosine (I-C) wobble base pair, or a combination thereof. In some embodiments, the at least one wobble base pair is to decrease the melting temperature of the second nucleic acid duplex. In some embodiments, the 5′ region of the first nucleic acid strand, the 3′ region of the first nucleic acid strand, and/or the third nucleic acid strand comprise one or more universal base. In some embodiments, the 5′ region, the 3′ region, or both, of the first nucleic acid strand comprise one or more universal base. The universal base can be, for example, hypoxanthine and derivatives thereof, inosine and derivatives thereof, azole carboxamide and derivatives thereof, nitroazole and derivatives thereof, phenyl C-ribonucleoside and derivatives thereof, naphthyl C-ribonucleoside and derivatives thereof, or a combination thereof. In some embodiments, the one or more universal base comprises inosine.

The central region of the first nucleic acid strand can comprise a sequence complementary to a target RNA. In some embodiments, the sequence complementary to the target RNA is 10-35 nucleosides in length, for example 10-21 nucleotides in length. In some embodiments, the second nucleic acid strand binds to 19-25 linked nucleotides in the central region of the first nucleic acid strand to form the first nucleic acid duplex. In some embodiments, the first nucleic acid duplex does not comprise a Dicer cleavage site. In some embodiments, the nucleic acid complex does not comprise a Dicer cleavage site.

The central region of the first nucleic acid strand can be linked to the 5′ region of the first nucleic acid strand via a 5′ connector. In some embodiments, the central region of the first nucleic acid strand is linked to the 3′ region of the first nucleic acid strand via a 3′ connector. In some embodiments, the 5′ connector, the 3′ connector, or both comprise a C₃ 3-carbon linker, a nucleotide, a modified nucleotide, or a exonuclease cleavage-resistant moiety, or a combination thereof. The modified nucleotide can be, for example, a 2′-O-methyl nucleotide or a 2′-F nucleotide. In some embodiments, the 5′ connector comprises, or is, a C₃ 3-carbon linker, 2′-O-methyl nucleotide, 2′-F nucleotide, a nucleotide with a phosphodiester 5′ and 3′ connection cleavable by an exonuclease when in a single stranded form, or a combination thereof. In some embodiments, the 3′ connector is a C₃ 3-carbon linker. In some embodiments, the 3′ connector comprises a C₃ 3-carbon linker, a nucleotide, a modified nucleotide, an exonuclease cleavage-resistant moiety when in a single stranded form, or a combination thereof.

The 3′ connector can comprise, or be, a 2′-O-methyl nucleotide, and the 2′-O-methyl nucleotide is optionally 2′-O-methyladenosine, 2′-O-methylguanosine, 2′-O-methyluridine, or 2′-O-methylcytidine. The second nucleic strand can be fully complementary to the central region of the first nucleic acid strand, thereby forming blunt ends at the 5′ and 3′ termini of the second nucleic acid strand in the first nucleic acid duplex. In some embodiments, the second nucleic acid strand does not have an overhand at 3′ terminus, or 5′ terminus, or both in the first nucleic acid duplex. The second nucleic acid strand can have a 3′ overhang, a 5′ overhang, or both in the first nucleic acid duplex. In some embodiments, the second nucleic acid strand has an 3′ overhang and the 3′ overhang is one to five nucleosides in length.

The 5′ terminus of the central region of the first nucleic acid strand, the 3′ terminus of the central region of the first nucleic acid strand, or both, can, for example, comprise at least one phosphorothioate internucleoside linkage. In some embodiments, each of the 5′ terminus of the central region of the first nucleic acid strand and the 3′ terminus of the central region of the first nucleic acid strand independently comprises one or more phosphorothioate internucleoside linkages. In some embodiments, the central region of the first nucleic acid strand does not comprise phosphorothioate internucleoside linkages except for the internucleoside linkage(s) between two or three nucleosides at the 5′ terminus, 3′ terminus, or both, of the central region. In some embodiments, at least 80%, at least 85%, at least 90%, or at least 95% of the nucleosides of one or more of (1) the central region of the first nucleic acid strand, (2) the 5′ region of the first nucleic strand, and (3) the 3′ region of the first nucleic strand are chemically modified. In some embodiments, at least 80%, at least 85%, at least 90%, or at least 95% of the nucleosides of one or more of the first nucleic acid strand, the second nucleic strand and the third nucleic strand are chemically modified. In some embodiments, at least 80%, at least 85%, at least 90%, at least 95%, or all of the nucleosides of the nucleic acid complex are chemically modified. In some embodiments, the chemical modifications are to resist nuclease degradation, to increase melting temperature (Tm), or both, of the nucleic acid complex. In some embodiments, at least 90%, at least 95%, or all of the nucleotides of the nucleic acid complex are non-DNA and non-RNA nucleotides.

In some embodiments, at most 5%, at most 10%, or at most 15% of the nucleosides of the second nucleic strand are locked nucleic acid (LNA). In some embodiments, about 10%-50% of the bases have a 2′-4′ bridging modifications. In some embodiments, about 10%-50% of the bases are LNA or analogues thereof. In some embodiments, about 10%-50% of the bases comprises 2′-O-methyl modification, 2′-F modification, or both. In some embodiments, less than 5%, less than 10%, less than 25%, less than 50% of the internucleoside linkages in the first nucleic acid strand are phosphorothioate internucleoside linkages. In some embodiments, the first nucleic acid strand does not comprise phosphorothioate internucleoside linkages. In some embodiments, the internucleoside linkages between (1) the one to three nucleotides adjacent to the 3′ of the 5′ connector, and/or (2) the one or two nucleotides adjacent to the 5′ of the 3′ connector, and/or (3) the one to three nucleotides adjacent to the 3′ of the 3′ connector, are phosphorothioate internucleoside linkages

The input nucleic acid strand can be, for example, a RNA. The target RNA can be, for example, a cellular RNA transcript. In some embodiments, the target RNA is an mRNA, an miRNA, a non-coding RNA, a viral RNA transcript, or a combination thereof. In some embodiments, the overhang of the second nucleic acid strand is capable of binding to the input nucleic acid strand to form a toehold, thereby causing the displacement of the second nucleic acid strand from the first nucleic acid strand. The overhang of the third nucleic acid strand can be, for example, 5 to 20 nucleosides in length, including 9 nucleotides in length. In some embodiments, all internucleoside linkages of the overhang of the third nucleic acid strand are phosphorothioate internucleoside linkages. In some embodiments, the 5′ terminus, the 3′ terminus, or both of the third nucleic acid strand comprises a terminal moiety. The terminal moiety can comprise, for example, a ligand, a fluorophore, a exonuclease, a fatty acid, a Cy3, an inverted dT attached to a tri-ethylene glycol, or a combination thereof.

The nucleic acid complex can comprise: a first nucleic acid strand comprising 20-60 linked nucleosides; a second nucleic acid strand binding to a first region of the first nucleic acid strand to form a first nucleic acid duplex; and a third nucleic acid strand binding to a second region of the first nucleic acid strand to form a second nucleic acid duplex, the third nucleic acid strand comprises a overhang, where the overhang is not complementary to the first nucleic acid strand and is capable of binding to an input nucleic acid strand to cause the displacement of the third nucleic acid strand from the first nucleic acid strand, the first region of the first nucleic acid strand is 3′ of the second region of the first nucleic acid strand, the third nucleic acid strand does not bind to any region of the first nucleic acid strand that is 3′ of the first region of the first nucleic acid strand, and the second nucleic acid duplex comprises at least one wobble base pair. The at least one wobble base pair can be, for example a guanine-uracil (G-U) wobble base pair, a hypoxanthine-uracil (I-U) wobble base pair, a hypoxanthine-adenine (I-A) wobble base pair, a hypoxanthine-cytosine (I-C) wobble base pair, or a combination thereof.

In some embodiments, the at least one wobble base pair is to decrease the melting temperature of the second nucleic acid duplex. In some embodiments, the second region of the first nucleic acid strand comprises one or more universal base. In some embodiments, a portion of the third nucleic acid strand that binds to the second region of the first nucleic acid strand comprises one or more universal base. The universal base can be, for example, hypoxanthine and derivatives thereof, inosine and derivatives thereof, azole carboxamide and derivatives thereof, nitroazole and derivatives thereof, phenyl C-ribonucleoside and derivatives thereof, naphthyl C-ribonucleoside and derivatives thereof, or a combination thereof. In some embodiments, the one or more universal base comprises inosine. In some embodiments, the nucleic acid complex the first region of the first nucleic acid strand comprises a sequence complementary to a target RNA, the sequence complementary to the target RNA is 10-35 nucleosides in length, for example 10-21 nucleotides in length.

In some embodiments, the second nucleic acid strand binds to 17-22 linked nucleotides in the first region of the first nucleic acid strand to form the first nucleic acid duplex. In some embodiments, the third nucleic acid strand binds to 10-30 linked nucleotides in the second region of the first nucleic acid strand to form the second nucleic acid duplex. In some embodiments, the third nucleic acid strand binds to about 14 linked nucleotides in the second region of the first nucleic acid strand to form the second nucleic acid duplex. In some embodiments, the first nucleic acid duplex does not comprise a Dicer cleavage site. In some embodiments, the nucleic acid complex does not comprise a Dicer cleavage site.

The first region of the first nucleic acid strand can be linked to the second region of the first nucleic acid strand via a linker. The linker can, for example, comprise a C₃ 3-carbon linker, a nucleotide, a modified nucleotide, or a exonuclease cleavage-resistant moiety, or a combination thereof. The modified nucleotide can be a 2′-O-methyl nucleotide or a 2′-F nucleotide. The 2′-O-methyl nucleotide can be 2′-O-methyladenosine, 2′-O-methylguanosine, 2′-O-methyluridine, or 2′-O-methylcytidine. The 2′-F nucleotide can be 2′-F adenosine, 2′-F guanosine, 2′-F uridine, or 2′-F cytidine. The 5′ terminus of the second nucleic acid strand can comprise a blocking moiety. The blocking moiety can comprise, or be, a fluorophore, an inverted-dT, a tri-ethylene-glycol, a fatty acid, a Cy3, or a combination thereof. The fluorophore can be attached to the 5′ terminus of the second nucleic strand via a phosphorothioate linkage.

In some embodiments, the first nucleic acid strand comprises a 3′ overhang in the first nucleic acid duplex. The 3′ overhang of the first nucleic acid can be, for example, one, two, or three nucleotides in length. In some embodiments, the 3′ overhang of the first nucleic acid comprises one or more phosphorothioate internucleoside linkages. In some embodiments, all of the internucleoside linkages in the 3′ overhang of the first nucleic acid are phosphorothioate internucleoside linkages. In some embodiments, the internucleoside linkage(s) between the last two, three or four nucleosides at the 3′ terminus of the first nucleic acid strand is phosphorothioate internucleoside linkage(s). In some embodiments, the first region of the first nucleic acid strand does not comprise phosphorothioate internucleoside linkages except for the internucleoside linkage(s) between the last two or three nucleosides at the 5′ terminus, 3′ terminus, or both. In some embodiments, the first region of the first nucleic acid strand does not comprise phosphorothioate internucleoside linkages except for the internucleoside linkage(s) between the last three nucleosides at the 5′ terminus and the last three nucleosides at 3′ terminus. In some embodiments, the second region of the first nucleic acid strand does not comprise phosphorothioate internucleoside linkages. In some embodiments, the second nucleic strand is fully complementary to the first region of the first nucleic acid strand, thereby forming no overhang at the 5′ and 3′ termini of the second nucleic acid strand in the first nucleic acid duplex. In some embodiments, the second nucleic acid strand does not have an overhang at 3′ terminus, or 5′ terminus, or both in the first nucleic acid duplex. In some embodiments, the second nucleic acid strand comprises one or more phosphorothioate internucleoside linkages

In some embodiments, the second nucleic acid strand does not comprise phosphorothioate internucleoside linkages except for the internucleoside linkage(s) between the last two to three nucleosides at the 5′ terminus and the last two to three nucleosides at 3′ terminus. In some embodiments, the internucleoside linkage(s) between the last two, three or four nucleosides at the 5′ terminus of the second nucleic acid strand, the 3′ terminus of the second nucleic acid strand, or both, are phosphorothioate internucleoside linkages. In some embodiments, the 5′ terminus of the third nucleic acid strand comprises at least one phosphorothioate internucleoside linkage. In some embodiments, the last two, three or four nucleosides at the 5′ terminus of the third nucleic acid strand are phosphorothioate internucleoside linkages. In some embodiments, less than 5%, less than 10%, less than 25%, less than 50% of the internucleoside linkages in the first nucleic acid strand are phosphorothioate internucleoside linkages. In some embodiments, the first nucleic acid strand comprises no more than two phosphorothioate internucleoside linkages, or does not comprise phosphorothioate internucleoside linkages.

In some embodiments, at least 50%, at least 60%, at least 70%, at least 80%, at least 85%, at least 90%, at least 95%, or all of the nucleosides of the first region of the first nucleic acid strand, the second region of the first nucleic strand, or both, are chemically modified. In some embodiments, at least 50%, at least 60%, at least 70%, at least 80%, at least 85%, at least 90%, at least 95%, or all of the nucleosides of one or more of the first nucleic acid strand, the second nucleic strand and the third nucleic strand are chemically modified. In some embodiments, at least 50%, at least 60%, at least 70%, at least 80%, at least 85%, at least 90%, at least 95%, or all of the nucleosides of the nucleic acid complex are chemically modified. In some embodiments, the chemical modifications are to resist nuclease degradation, to increase melting temperature (Tm), or both, of the nucleic acid complex. In some embodiments, at least 90%, at least 95%, or all of the nucleotides of the nucleic acid complex are non-DNA and non-RNA nucleotides. In some embodiments, at most 5%, at most 10%, or at most 15% of the nucleosides of the second nucleic strand are LNA. In some embodiments, about 10%-50% of the bases of the nucleic acid complex have a 2′-4′ bridging modifications. In some embodiments, about 10%-50% of the bases of the nucleic acid complex are LNA or analogues thereof. In some embodiments, about 10%-50% of the bases of the nucleic acid complex comprises 2′-O-methyl modification, 2′-F modification, or both.

The input nucleic acid strand can be, for example, a RNA. The target RNA can be, for example, a cellular RNA transcript. In some embodiments, the target RNA is an mRNA, an miRNA, a non-coding RNA, a viral RNA transcript, or a combination thereof. In some embodiments, the overhang of the second nucleic acid strand is capable of binding to the input nucleic acid strand to form a toehold, thereby causing the displacement of the second nucleic acid strand from the first nucleic acid strand. In some embodiments, the overhang of the third nucleic acid strand is 5 to 20 nucleosides in length, for example 12 nucleotides in length.

In some embodiments, all internucleoside linkages of the overhang of the third nucleic acid strand are phosphorothioate internucleoside linkages. In some embodiments, the 5′ terminus, the 3′ terminus, or both of the third nucleic acid strand comprises a terminal moiety. The terminal moiety can, for example, comprise a ligand, a fluorophore, a exonuclease, a fatty acid, a Cy3, an inverted dT attached to a tri-ethylene glycol, or a combination thereof.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 illustrates a schematic representation of two non-limiting exemplary nucleic acid complex constructs.

FIG. 2 illustrates a schematic representation of a non-limiting exemplary nucleic acid complex construct.

FIG. 3 illustrates a schematic representation of two non-limiting exemplary nucleic acid complex constructs.

FIG. 4 illustrates a schematic representation of a sensor nucleic acid strand, a core nucleic acid strand and a passenger nucleic acid strand of a non-limiting exemplary nucleic acid complex.

FIG. 5 illustrates a schematic representation of a non-limiting exemplary nucleic acid complex construct with regions for screening highlighted in yellow.

FIG. 6 is a schematic diagram showing the formation of an active RNAi duplex following the displacement of a sensor nucleic acid strand from a core nucleic acid strand and the degradation of the core nucleic acid strand overhangs.

FIG. 7A and FIG. 7B show sequence diagrams of two non-limiting exemplary nucleic acid complex constructs having the same passenger strand but different core strand. Core strand v3c1: from 5′ to 3′ SEQ ID NO: 3-5 joined by a C3 spacer; Passenger strand v3p1: SEQ ID NO: 2; Core strand v3c5: SEQ ID NO: 11; Passenger strand 1: SEQ ID NO: 2.

FIG. 8 show sequence diagrams of two positive control constructs. HTT Guide 1: SEQ ID NO: 21; HTT Pass 1: SEQ ID NO: 22; HTT Guide 2: SEQ ID NO: 23; HTT Pass 2: SEQ ID NO: 24.

FIG. 9 shows various siRNA complex variants with different passenger strand (V3P1, V3P2, V3P3, V3P5, V3P5, V3P6, V3P7, V3P8, and V3P9) assembled with an exemplary core strand (v3c1 which include two C3 linkers) shown in FIG. 7A and used in target protein expression shown in FIG. 10.

FIG. 10 shows a graphic representation of the target protein expression data generated using the siRNA complex deign variants shown in FIG. 9.

FIG. 11 shows various siRNA complex variants with different passenger strand (V3P1, V3P2, V3P3, V3P5, V3P5, V3P6, V3P7, V3P8, and V3P9) assembled with an exemplary core strand (v3c5 which does not include a C3 linker) shown in FIG. 7B.

FIG. 12 shows a graphic representation of the target protein expression data generated using the siRNA complex variants shown in FIG. 11.

FIG. 13A and FIG. 13B show sequence diagrams of various exemplary nucleic acid complex constructs each having the same passenger strand (Passenger strand 1) and the same sensor strand (Mir23 Sensor 1) but a different core strand (Core strand v3c1, Core strand v3c2, Core strand v3c3, Core strand v3c4, Core strand v3c5, and Core strand v3c6, which are referred to as C1, C2, C3, C4, C5, C6, respectively, in FIGS. 15-16 and description thereof). The sequences shown in FIG. 13A and 13B are listed in Table 1.

FIG. 14 shows non-denaturing polyacrylamide gel (PAGE) of various nucleic acid complex constructs.

FIG. 15 shows the RNAi activity of two-stranded assemblies each having the same passenger strand v3p1 and a different core strand (C1, C2, C3, C4, C5, and C6) at different concentrations.

FIG. 16 shows the RNAi activity of three-stranded assemblies each having the same passenger strand v3p1, the same sensor strand (Mir23 sensor 1), and a different core strand (C1, C2, C3, C4, C5, and C6) at three different concentrations.

FIG. 17 shows sequence diagrams of a non-limiting exemplary nucleic acid complex construct disclosed herein (top: V3C3a) and a partially modified nucleic acid complex (bottom: GIC1S1). The sequences shown in FIG. 17 are listed in Table 2.

FIG. 18 shows the RNAi activity of the exemplary two-stranded nucleic acid complex constructs (V3C3a siRNA) and three-stranded nucleic acid complex constructs (V3C3a and V3C3b) in comparison with the partially modified two-stranded construct (G1C1 siRNA) and the partially modified three-stranded constructs (GIC1S1) shown in FIG. 17 at three different concentrations.

FIG. 19 shows sequence diagrams of three non-limiting exemplary nucleic acid complex constructs. Alt anp sens1: SEQ ID NO: 33; Alt anp-calc core 1: SEQ ID NO: 34; Alt anp sens2: SEQ ID NO: 35; Alt mus-calc core2: SEQ ID NO: 36; Alt mus-calc core 3: SEQ ID NO: 37. Calc V3P3 passenger: SEQ ID NO: 13.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

RNA interference (RNAi) is an intrinsic cellular mechanism conserved in most eukaryotes, that helps to regulate the expression of genes critical to cell fate determination, differentiation, survival and defense from viral infection. Researchers have exploited this natural mechanism by designing synthetic double-stranded RNA for sequence-specific gene silencing. Emerging developments in the field of dynamic nuclei acid nanotechnology and biomolecular computing also offer a conceptual approach to design programmable RNAi agents. However, challenges still remain in developing targeted RNAi therapy that can use nuclei acid logic switches to sense RNA transcripts (such as mRNAs and miRNAs) in order to restrict RNA silencing to specific populations of disease-related cells and spare normal tissues from toxic side effects. Significant challenges include poorly suppressed background drug activity, weak activated state drug potency, input and output sequence overlap, high design complexity, short lifetimes (<24 hours) and high required device concentrations (>10 nM).

Provided herein include signal activatable small interfering RNA (siRNA) complexes, components, compositions, and related methods and systems. The signal activatable siRNA complex can switch from an inactivated state to an activated state when triggered by a complementary binding of an input nucleic acid strand (e.g., a disease biomarker gene specific to disease-related cells) to the siRNA complex, thereby activating the RNA interference activity of the siRNA complex to target a specific target RNA (e.g., a RNA to be silenced). The nucleic acid complexes herein described can mediate conditionally activated RNA interference activity to silence target RNA in specific populations of disease-related cells with improved potency at a low concentration as well as improved specificity that can reduce off-target effects.

Disclosed herein includes a nucleic acid complex. The nucleic acid complex comprises a first nucleic acid strand (e.g., core nucleic acid strand). a second nucleic acid strand (e.g., passenger nucleic acid strand) binding to a central region of the first nucleic acid strand to form a first nucleic acid duplex (e.g., RNAi duplex), and a third nucleic acid strand (e.g., sensor nucleic acid strand) binding to a 5′ region and a 3′ region of the core nucleic acid strand to form a second nucleic acid duplex (e.g., sensor duplex). The sensor nucleic acid strand comprises a overhang, wherein the overhang is not complementary to the first nucleic acid strand and is capable of binding to an input nucleic acid strand to cause the displacement of the sensor nucleic acid strand from the core nucleic acid strand. The sensor duplex can comprise at least one wobble base pair. The core nucleic acid strand can comprise 20-70 linked nucleosides. The central region of the core nucleic acid strand can comprise a sequence complementary to a target RNA. The sequence complementary to a target RNA can be 10-35 nucleosides in length.

Disclosed herein includes another nucleic acid complex. The nucleic acid complex comprises a first nucleic acid strand (e.g., core nucleic acid strand), a second nucleic acid strand (e.g., passenger nucleic acid strand) binding to a first region of the core nucleic acid strand to form a first nucleic acid duplex (e.g., RNAi duplex), and a third nucleic acid strand (e.g., sensor nucleic acid strand) binding to a second region of the core nucleic acid strand to form a second nucleic acid duplex (e.g., sensor duplex). The sensor nucleic acid strand comprises an overhang that is not complementary to the core nucleic acid strand and is capable of binding to an input nucleic acid strand to cause the displacement of the third nucleic acid strand from the first nucleic acid strand. The first region of the core nucleic acid strand is 3′ of the second region of the core nucleic acid strand. The sensor nucleic acid strand does not bind to any region of the core nucleic acid strand that is 3′ of the first region of the core nucleic acid strand. The sensor duplex can comprise at least one wobble base pair. The core nucleic acid strand can comprise 20-60 linked nucleosides. The first region of the core nucleic acid strand can comprise a sequence complementary to a target RNA. The sequence complementary to a target RNA can be 10-35 nucleosides in length.

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded or multi-stranded (e.g., double-stranded or triple-stranded). “mRNA” or “messenger RNA” is single-stranded RNA molecule that is complementary to one of the DNA strands of a gene. “miRNA” or “microRNA” is a small single-stranded non-coding RNA molecule that functions in RNA silencing and post-transcriptional regulation of gene expression.

The term “RNA analog” refers to an polynucleotide having at least one altered or modified nucleotide as compared to a corresponding unaltered or unmodified RNA. The nucleotide can retain the same or similar nature or function as the corresponding unaltered or unmodified RNA such as forming base pairs.

A single-stranded polynucleotide has a 5′ terminus or 5′ end and a 3′ terminus or 3′ end The terms “5′ end” “5′ terminus” and “3′ end” “3′ terminus” of a single-stranded polynucleotide indicate the terminal residues of the single-stranded polynucleotide and are distinguished based on the nature of the free group on each extremity. The 5′-terminus of a single-stranded polynucleotide designates the terminal residue of the single-stranded polynucleotide that has the fifth carbon in the sugar-ring of the deoxyribose or ribose at its terminus (5′ terminus). The 3′-terminus of a single-stranded polynucleotide designates the residue terminating at the hydroxyl group of the third carbon in the sugar-ring of the nucleotide or nucleoside at its terminus (3′ terminus). The 5′ terminus and 3′ terminus in various cases can be modified chemically or biologically e.g., by the addition of functional groups or other compounds as will be understood by the skilled person.

As used herein, the terms “complementary binding” and “bind complementarily” mean that two single strands are base paired to each other to form nucleic acid duplex or double-stranded nucleic acid. The term “base pair” as used herein indicates formation of hydrogen bonds between base pairs on opposite complementary polynucleotide strands or sequences following the Watson-Crick base pairing rule. For example, in the canonical Watson-Crick DNA base pairing, adenine (A) forms a base pair with thymine (T) and guanine (G) forms a base pair with cytosine (C). In RNA base paring, adenine (A) forms a base pair with uracil (U) and guanine (G) forms a base pair with cytosine (C). A certain percentage of mismatches between the two single strands are allowed as long as a stable double-stranded duplex can be formed. In some embodiments, the two strands that bind complementarily can have a mismatches can be, about, be at most, or be at most bout 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%.

As used herein, the terms “RNA interference”, “RNA interfering”, and “RNAi” refer to a selective intracellular degradation of RNA. RNAi can occur in cells naturally to remove foreign RNAs (e.g., viral RNAs). Natural RNAi proceeds via fragments cleaved from free dsRNA which direct the degradative mechanism to other similar RNA sequences. RNAi can also be initiated non-naturally, for example, to silence the expression of target genes.

As used herein, the terms “small interfering RNA” and “siRNA” refer to an RNA or RNA analog capable of reducing or inhibiting expression of a gene or a target gene when the siRNA is activated in the same cell as the target gene. The siRNA used herein can comprise naturally occurring nucleic acid bases and/or chemically modified nucleic acid bases (RNA analogs).

### Nucleic Acid Complexes

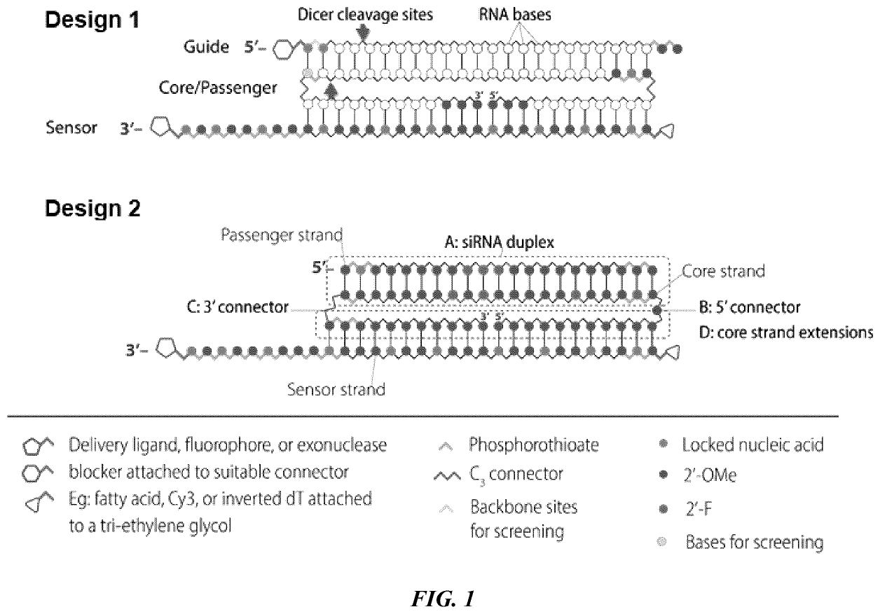

Provided herein include a nucleic acid complex that can be conditionally activated upon a complementary binding to an input nucleic acid strand (e.g., a mRNA of a disease biomarker gene specific to a target cell (e.g., disease-related cells)) through a sequence in a sensor nucleic acid strand of the nucleic acid complex. The activated nucleic acid complex can release a potent RNAi duplex formed by a core nucleic acid strand and a passenger nucleic acid strand, which can specifically inhibit or silence a target RNA. The target RNA can have a sequence independent from the input nucleic acid strand. The target RNA can be from a gene that is different from the gene that the input nucleic acid strand is from. In some embodiments, the target RNA is from a gene that is the same as the gene that the input nucleic acid strand is from. FIGS. 1-3 illustrates schematic representations of non-limiting exemplary nucleic acid complex constructs.

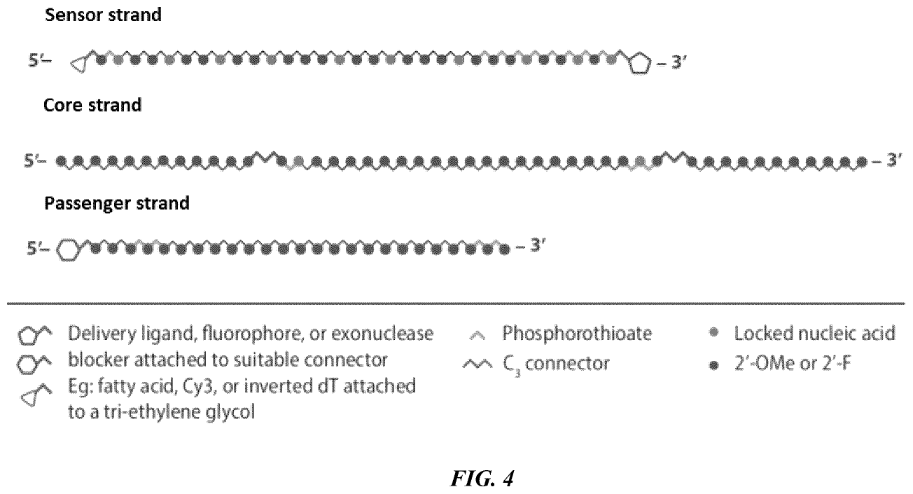

In some embodiments, the nucleic acid complexes described herein comprise a core nucleic acid strand, a passenger nucleic acid strand, and a sensor nucleic acid strand as shown in a non-limiting embodiment of FIG. 4. These three strands can base-pair with one another to form, for example, a RNAi duplex and a sensor duplex. One or more of the core nucleic acid strand, the passenger nucleic acid strand, and the sensor nucleic acid strand can be RNA analogs comprising modified nucleotides.

The term “nucleic acid duplex” as used herein refers to two single-stranded polynucleotides bound to each other through complementarily binding. The nucleic acid duplex can form a helical structure, such as a double-stranded RNA molecule, which is maintained largely by non-covalent bonding of base pairs between the two single-stranded polynucleotides and by base stacking interactions.

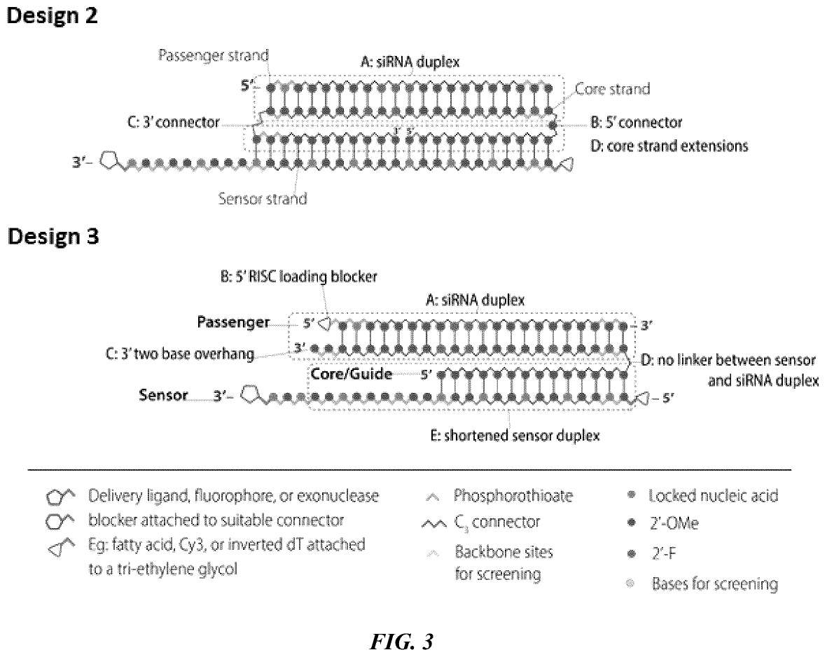

The core nucleic acid strand of a nucleic acid complex herein described can comprise a 5′ region, a 3′ region, and a central region between the 5′ region and the 3′ region (see e.g., in FIG. 1 and FIG. 3). The central region of the core nucleic acid strand can be linked to the 5′ region and/or the 3′ region of the core nucleic acid strand via a connector. In some embodiments, the central region of the core nucleic acid strand is linked the 5′ region of the core nucleic acid strand via a 5′ connector. In some embodiments, the central region of the core nucleic acid strand is linked to the 3′ region of the core nucleic acid strand via a 3′ connector. The central region of the core nucleic acids strand is complementarily bound to the passenger nucleic acid strand to form a RNAi duplex. Not the entire sequence of the core nucleic acid strand is complementarily bound to the passenger nucleic acid strand. For example, the 5′ region and the 3′ region of the core nucleic acid strand is not complementarily bound to the passenger nucleic acid strand.

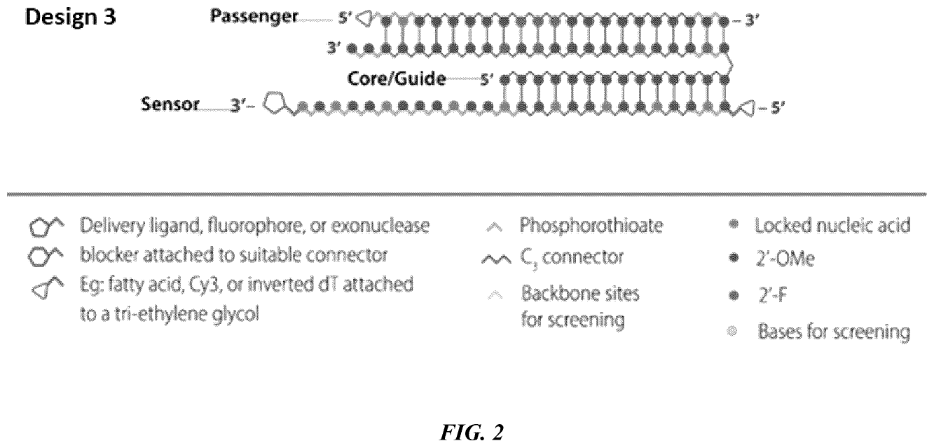

The core nucleic acid strand can comprise a first region and a second region and the first region is at the 3′ direction of the second region (see e.g., in FIGS. 2-3). In other words, the first region is at the 3′ end of the core nucleic acid strand and the second region is at the 5′ end of the core nucleic acid strand. The first region of the core nucleic acid strand can be linked to the second region of the core nucleic acid strand via a connector, which can also be referred to as a 5′ connector. The 5′ connector can be a normal phosphodiester internucleoside linkage connecting two adjacent nucleotides. In some embodiments, the core nucleic acid strand only comprises one connector (e.g., 5′ connector) and does not comprise a 3′ connector. The first region of the core nucleic acids strand is complementarily bound to the passenger nucleic acid strand to form a RNAi duplex. Not the entire sequence of the core nucleic acid strand is complementarily bound to the passenger nucleic acid strand. For example, the second region of the core nucleic acid strand is not complementarily bound to the passenger nucleic acid strand. In some embodiments, the first region of the core nucleic acid strand is fully complementary to the passenger nucleic acid strand, thereby forming a RNAi duplex having a blunt end with no overhang at the 5′ and 3′ termini of the first region of the core nucleic acid strand. In some embodiments, the core nucleic acid strand of the RNAi duplex has a short overhang at the 3′ terminus (e.g., one, two, or three nucleosides), but the 3′ overhang does not extend back into the middle of the sensor duplex to bind with the sensor nucleic acid strand (see e.g., in FIGS. 2-3). In some embodiments, the core nucleic acid strand does not have any region at the 3′ of the first region of the core nucleic acid strand.

The core nucleic acid strand (e.g., the central region in Design 1 and Design 2or the first region in Design 3) can comprise a sequence complementary to a target nucleic acid (e.g., a RNA to be silenced). The core nucleic acid strand of the nucleic acid complex acts as a guide strand (antisense strand) and is used to base pair with a target RNA. The passenger nucleic acid strand can therefore comprise a sequence homologous to the same target nucleic acid.

Upon activation of the nucleic acid complex (e.g., binding to an input nucleic acid strand), the released RNAi duplex can complementarily bind a target nucleic acid through the binding between the target nucleic acid and the core nucleic acid strand. In some embodiments, the sequence complementary to a target RNA in the core nucleic acid strand can be about 10-35 nucleosides in length. In some embodiments, the core nucleic acid strand comprises 20-70 linked nucleosides. In some embodiments, the core nucleic acid strand comprises 20-60 linked nucleosides.

In some embodiments wherein the core nucleic acid strand comprises a 5′ region, a central region and a 3′ region (e.g., in FIG. 1 and FIG. 2), the sensor nucleic acid strand is complementarily bound to the 5′ region and the 3′ region of the core nucleic acid strand to form a sensor duplex. The sensor nucleic acid strand does not bind to the central region of the core nucleic acid strand nor the passenger nucleic acid strand.

In some embodiments wherein the core nucleic acid strand comprises a first region and a second region (e.g., in FIGS. 2-3), the sensor nucleic acid strand is complementarily bound to the second region of the core nucleic acid strand to form a sensor duplex. The sensor nucleic acid strand does not bind to the first region of the core nucleic acid strand nor any region of the core nucleic acid strand that is 3′ of the first region of the core nucleic acid strand. The sensor nucleic acid strand also does not bind to the passenger nucleic acid strand.

The sensor nucleic acid strand can comprise an overhang. The term “overhang” as used herein refers to a stretch of unpaired nucleotides that protrudes at one of the ends of a double-stranded polynucleotide (e.g., a duplex). An overhang can be on either strand of the polynucleotide and can be included at either the 3′ terminus of the strand (3′ overhang) or at the 5′ terminus of the strand (5′ overhang). The overhang can be at the 3′ terminus of the sensor nucleic acid strand. The overhang of the sensor nucleic acid strand does not bind to any region of the core nucleic acid strand.

The sensor nucleic acid strand can comprise a sequence capable of binding to an input nucleic acid strand (e.g., a mRNA of a disease biomarker gene specific to a target cell, including a disease-related cell). Upon activation, the binding of the sensor nucleic acid strand to the input nucleic acid strand can cause displacement and subsequent release of the sensor nucleic acid strand from the core nucleic acid strand, thereby releasing the potent RNAi duplex and switching on the RNA interfering activity of the RNAi duplex. In the absence of an input nucleic acid strand or a detectable amount of the input nucleic acid strand, the nucleic acid complex herein described remains in an inactivated state (switched off) and the displacement of the sensor nucleic acid strand from the core nucleic acid strand does not take place. Therefore, the input nucleic acid strand can act as a trigger to activate (switch on) the RNA interfering activity of the nucleic acid complex (e.g., RNAi duplex).

The length of the RNAi duplex of the nucleic acid complex herein described can vary in different embodiments. In some embodiments, the length of the RNAi duplex can be 10-35 nucleotides, optionally 10-30 nucleotides. For example, the length of the RNAi duplex can be, 10, 11, 12, 13, 14, 15, 16, 17, 18, 19, 20, 21, 22, 23, 24, 25, 26, 27, 28, 29, 30, 31, 32, 33, 34, 35, a range of any two of these values, nucleotides. In some embodiments, the length of the RNAi duplex can be 19-25 nucleotides. In some embodiments, the length of the RNAi duplex can be 17-22 nucleotides.

The length of the sensor duplex of the nucleic acid complex herein described can vary in different embodiments. In some embodiments, the length of the sensor duplex can be 10-35 nucleotides, optionally 10-30 nucleotides. For example, the length of the sensor duplex can be, 10, 11, 12, 13, 14, 15, 16, 17, 18, 19, 20, 21, 22, 23, 24, 25, 26, 27, 28, 29, 30, 31, 32, 33, 34, 35, a range of any two of these values, nucleotides. In some embodiments, the length of the sensor duplex is about 14 nucleotides. In some embodiments, the sensor duplex has a relatively short length with respect to the RNAi duplex.

In some embodiments, there is no linker molecule between a sensor duplex and a RNAi duplex of a nucleic acid complex except for the normal phosphodiester linkage connecting two adjacent nucleosides each located at a terminus of one of the two duplexes.

In some embodiments, the sensor duplex formed by a portion of a core nucleic acid strand and a portion of a sensor nucleic acid strand can comprise one or more wobble base pair or mismatch. The term “wobble base pair” as used herein refers to a base pairing between two nucleotides in a nucleic acid duplex that does not follow Watson-Crick base pair rules. Wobble base pairs can include guanine-uracil (G-U), hypoxanthine-uracil (I-U), hypoxanthine-adenine (I-A), and hypoxanthine-cytosine (I-C). The denotation “I” refers to hypoxanthine which forms an inosine when attached to a ribose ring. In some embodiments, the wobble base pair can be introduced by, for example, substituting C for U, A for G, and/or A, U, or G for I. The wobble base pairs may be used every about 4-8 nucleotides. In some embodiments, a wobble base pair forms between two naturally occurring nucleotide bases such as G-U. In some embodiments, a wobble base pair forms between a naturally occurring nucleotide base (e.g., A, U, or C) and a universal base (e.g., I).

In some embodiments wherein the core nucleic acid strand comprises a 5′ region, a central region and a 3′ region (e.g., Design 2 in FIGS. 1 and 3), the wobble base pairs can occur between the 5′ region of the core nucleic acid strand and the sensor nucleic acid strand. In some embodiments, the wobble base pairs can occur between the 3′ region of the core nucleic acid strand and the sensor nucleic acid strand. In some embodiments, the wobble base pairs can occur between both the 5′ region and the 3′ region of the core nucleic acid strand and the sensor nucleic acid strand.

In some embodiments wherein the core nucleic acid strand comprises a first region and a second region (e.g., Design 3 in FIGS. 2-3), the wobble base pairs can occur between the second region of the core nucleic acid strand and the sensor nucleic acid strand.

The bond strength or base pair strength in a portion of the sensor duplex can be reduced due to the presence of the wobble base pairs (e.g., G-U, I-U, I-A, and/or I-C). Therefore, in some embodiments, the wobble base pairs in the sensor duplex can decrease the thermodynamic stability of the sensor duplex, such as to lower the melting temperature of the sensor duplex, thereby promoting the toehold-mediated displacement of the sensor nucleic acid strand from the core nucleic acid strand triggered by an input nucleic acid strand.

The nucleic acid complexes herein described can be synthesized using standard methods for oligonucleotide synthesis well-known in the art including, for example, Oligonucleotide Synthesis by Herdewijin, Piet (2005) and Modified oligonucleotides: Synthesis and Strategy for Users, by Verma and Eckstein, Annul Rev. Biochem. (1998): 67:99-134, the contents of which are incorporated herein by reference in their entirety. The synthesized nucleic acid complexes can be allowed to form its secondary structure under a desirable physiological condition as will be apparent to a skilled artisan. The formed secondary structure can be tested using standard methods known in the art such as chemical mapping, NMR, or computational simulations. The nucleic acid complex construct can be further modified, according to the test result, by introducing or removing chemical modifications or mismatches, as necessary, until the desired structure is obtained.

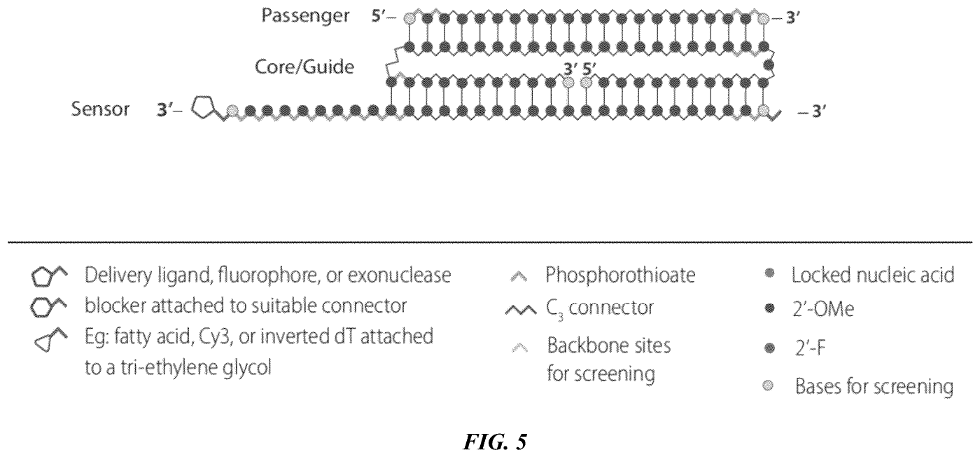

Suitable software suites can be used to aid in the design and analysis of nucleic acid structures. For example, Nupack can be used to check the formation of the duplexes and to rank the thermodynamic stability of the duplexes. Oligonucleotide design tools can be used to optimize the placement of LNA modifications. Any of the regions of one or more of the strands in a nucleic acid complex herein described can be screened for an input nucleic acid sequence, a target nucleic acid sequence and/or chemical modifications herein described. For example, FIG. 5 illustrates a schematic representation of a non-limiting exemplary nucleic acid complex construct, highlighting in yellow the terminal bases that can be screened for chemical modifications such as LNA placements and other nucleotide analogs herein described.

The nucleic acid complexes generated using the methods herein described can be delivered to a target site, in vivo, ex vivo or in vitro, to modulate a target RNA. For example, a cell at the target site comprising a target RNA can be contacted with the nucleic acid complex herein described. Upon detection of an input nucleic acid strand, an input strand can bind to the overhang of the sensor nucleic acid strand to cause displacement of the sensor nucleic acid strand from the core nucleic acid strand to release the sequence complementary to the target RNA into the cell, thereby modulating the target RNA. The nucleic acid complexes generated can also be used to treat a disease or a condition in a subject or an individual. For example, the nucleic acid complex generated herein can be administered to the cells, tissues, and/or organs of a subject in need thereof in an effective amount via any suitable local or systemic administration route. Upon detection of an input nucleic acid strand, the input nucleic acid strand can bind to the overhang of the sensor nucleic acid strand to cause displacement of the sensor nucleic acid strand from the core nucleic acid strand to release the sequence complementary to a target RNA, thereby reducing the activity of the target RNA or protein expression from the target RNA in the subject to treat the disease or condition. Various delivery systems can be employed for delivering the nucleic acid complex herein described such as antibody conjugates, micelles, natural polysaccharides, peptides, synthetic cationic polymers, microparticles, lipid-based nanovectors among others as will be apparent to a skilled artisan.

### RNA Interference (RNAi)

Described herein are nucleic acid complexes that can be conditionally activated (e.g., via a signal for the presence of a mRNA of a gene specific for a target cell) to switch from an assembled, inactivated state to an activated state to act on (e.g., degrade or inhibit) a specific target nucleic acid in response to the detection of an input nucleic acid (e.g., a nucleic acid sequence specific to a target cell, including a disease-related cell) having a sequence complementary to a sequence in the sensor nucleic acid strand of a nucleic acid complex.

In the assembled, inactivated configuration, the sensor nucleic acid strand of the nucleic acid complex inhibits enzymatic processing of the RNAi duplex, thereby keeping RNAi activity switched off. In the event that an input nucleic acid strand complementary to the sensor nucleic acid strand of a nucleic acid complex is present, the input nucleic acid strand can activate the nucleic acid complex by inducing separation of the sensor nucleic acid strand from the core nucleic acid strand via toehold mediated strand displacement. Displacement can start from a toehold formed at the 3′ or 5′ terminus of the sensor nucleic acid strand (e.g., a 5′ toehold or a 3′ toehold) through a complementary binding between the input nucleic acid strand and an overhang of the sensor nucleic acid strand.

After removal of the sensor nucleic acid strand, the region of the core nucleic acid strand that is not complementary bound to the passenger nucleic acid strand become an overhang region that can be degraded by nucleases (e.g., exonuclease). For example, in some embodiments (e.g., in FIG. 1), the 3′ and 5′ region of the core nucleic acid strand become 3′ and 5′ overhangs. In some other embodiments (e.g., in FIGS. 2-3), the second region of the core nucleic acid strand becomes a 5′ overhang.

This degradation stops at the 3′ end and/or 5′ end of the RNAi duplex due to the presence of chemically modified nucleotides and/or exonuclease cleavage-resistance moieties, thus rendering an active RNAi duplex for further endonuclease processing if needed and RNA-induced silencing complex (RISC) loading.

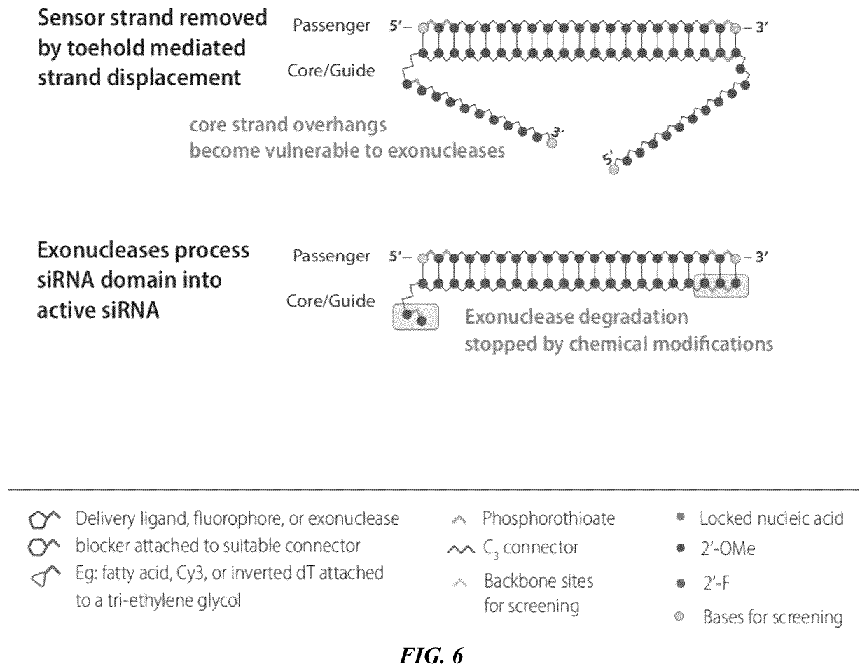

FIG. 6 is a non-limiting schematic diagram showing the formation of an active RNAi duplex following the displacement of a sensor nucleic acid strand from a core nucleic acid strand and the degradation of the core nucleic acid strand overhangs.

RISC is a multiprotein complex that incorporates one strand of a siRNA or miRNA and uses the siRNA or miRNA as a template for recognizing complementary target nucleic acid. Once a target nucleic acid is identified, RISC activates RNase (e.g., Argonaute) and inhibits the target nucleic acid by cleavage. In some embodiments, Dicer is not required for loading the RNAi duplex into RISC.

The passenger nucleic acid strand is then discarded, while the core nucleic acid strand is incorporated into RICS. The core nucleic acid strand of the nucleic acid complex disclosed herein acts as a guide strand (antisense strand) and is used to base pair with a target RNA. The passenger nucleic acid strand acts as a protecting strand prior to the loading of the core nucleic acid strand into RICS. RICS uses the incorporated core nucleic acid strand as a template for recognizing a target RNA that has complementary sequence to the core nucleic acid strand, particularly the central region of the core nucleic acid strand. Upon binding to the target RNA, the catalytic component of RICS, Argonaute, is activated which can degrade the bound target RNA. The target RNA can be degraded or the translation of the target RNA can be inhibited.

In some embodiments, the nucleic acid complexes generated herein do not have a dicer cleavage site, and therefore the RNAi interference mediated by the nucleic acid complexes can bypass Dicer-mediated cleavage. As will be apparent to a skilled artisan, Dicer is an endoribonuclease in the RNAse III family that can initiate the RNAi pathway by cleaving double-stranded RNA (dsRNA) molecule into short fragments of dsRNAs about 20-25 nucleotides in length. In some embodiments, the nucleic acid complexes generated herein differentiate from the conditionally activated small interfering RNAs (Cond-siRNAs) disclosed in the related international application published as WO 2020/033938 in that the nucleic acid complexes generated herein can bypass the Dicer processing.

In some embodiments, the nucleic acid complexes generated herein have structural features that discourage the Dicer binding. In some embodiments, the RNAi duplex does not create a Dicer substrate. For example, the RNAi duplex formed by the passenger nucleic acid strand and the core nucleic acid strand do not have a 3′ and/or 5′ overhang, but instead forming a blunt end that can render the passenger nucleic acid strand unfavorable for Dicer binding. In some embodiments, the passenger nucleic acid strand has about 17-22 nucleotides in length, making it short enough to bypass Dicer cleavage. In some embodiments, the passenger nucleic acid strand does not have G/C rich bases to the 3′ and/or 5′ end of the passenger nucleic acid strand. In some embodiments, the passenger nucleic acid strand are attached to a terminal moiety to avoid Dicer binding.

Upon activation, the nucleic acid complex generated herein can inhibit a target nucleic acid in target cells, therefore resulting in a reduction or loss of expression of the target nucleic acid in the target cells. The target cells are cells associated or related to a disease or disorder. The term “associated to” “related to” as used herein refers to a relation between the cells and the disease or condition such that the occurrence of a disease or condition is accompanied by the occurrence of the target cells, which includes but is not limited to a cause-effect relation and sign/symptoms-disease relation. The target cells used herein typically have a detectable expression of an input nucleic acid.

In some embodiments, the expression of a target nucleic acid in target cells is inhibited about, at least, at least about, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any of these values.

As used herein, inhibition of gene expression refers to the absence or observable decrease in the level of protein and/or mRNA product from a target gene in target cells. The degree of inhibition can be evaluated by examination of the expression level of the target gene as demonstrated in the examples.

Gene expression and/or the inhibition of target gene expression can be determined by use of a reporter or drug resistance gene whose protein product is easily assayed. Exemplary reporter genes include, but no limiting to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Quantitation of the amount of gene expression allows one to determine a degree of inhibition as compared to cells not treated with the nucleic acid complexes or treated with a negative or positive control. Various biochemical techniques may be employed as will be apparent to a skilled artisan such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

In some embodiments, the nucleic acid complexes generated herein exhibit improved switching performance and reduced off-target effects. The nucleic acid complexes generated herein can have a reduced unwanted RNAi activity when the nucleic acid complexes are in an inactivated state (switched off) and an enhanced RNAi activity when the nucleic acid complexes are activated upon detection of an input nucleic acid strand.

In some embodiments, the expression of a target nucleic acid in non-target cells (e.g., cells not having an input nucleic acid strand) is inhibited about, at most, or at most about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any of these values. Non-target cells can comprise cells of the subject other than target cells.

In some embodiments, the nucleic acid complexes generated herein have an enhanced potency, thus capable of evoking an RNAi activity at low concentrations. Nonspecific, off-target effects and toxicity (e.g., undesired proinflammatory responses) can be minimized by using low concentrations of the nucleic acid complexes.

The concentration of the nucleic acid complexes generated herein can vary in different embodiments. In some embodiments, the nucleic acid complexes generated herein can be provided at a concentration of, about, at most, or at most about, 0.001 nM, 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3 nM, 0.4 nM, 0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0 nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, 11 nM, 12 nM, 13 nM, 14 nM, 15 nM, 16 nM, 17 nM, 18 nM, 19 nM, 20 nM, 30 nM, 40 nM, 50 nM, or a number or a range between any two of these values. For example, the nucleic acid complexes generated herein can be provided at a concentration between about 0.1-10 nM, preferably between about 0.1-1.0 nM. In some embodiments, the nucleic acid complex generated herein has a transfection concentration at about 0.1 nM or lower.

The nucleic acid complex herein described can allow lasting and consistently potent inhibition effects at low concentrations. For example, the nucleic acid complex can remain active for an extended period of time such as 1 hour, 2 hours, 6 hours, 12 hours, 24 hours, 36 hours, 48 hours, 60 hours, 72 hours, 84 hours, 96 hours, 5 days, 6 days, 7 days, two weeks, or a number or a range between any of these values, or more. In some embodiments, the nucleic acid complex can remain active for at least 12 hours, at least 24 hours, at least 36 hours, at least 48 hours, at least 60 hours, at least 72 hours, at least 84 hours, or at least 96 hours. In some embodiments, the nucleic acid complex can remain active for up to 30 days, up to 60 days, or up to 90 days.

### Chemical Modification

The nucleic acid strands (the core nucleic acid strand, the passenger nucleic acid strand, and/or the sensor nucleic acid strand) comprised in the nucleic acid complexes herein described can be a non-standard, modified nucleic acid strand comprising non-standard, modified nucleotides (nucleotide analog) or non-standard, modified nucleosides (nucleoside analog). The term “nucleotide analog” or “modified nucleotide” refers to a non-standard nucleotide comprising one or more modifications (e.g., chemical modifications), including non-naturally occurring ribonucleotides or deoxyribonucleotides. The term “nucleoside analog” or “modified nucleoside” refers to a non-standard nucleoside comprising one or more modification (e.g., chemical modification), including non-naturally occurring nucleosides other than cytidine, uridine, adenosine, guanosine, and thymidine. The modified nucleoside can be a modified nucleotide without a phosphate group. The chemical modifications can include replacement of one or more atoms or moieties with a different atom or a different moiety or functional group (e.g., methyl group, and hydroxyl group).

The modifications are introduced to alter certain chemical properties of the nucleotide/nucleoside such as to increase or decrease thermodynamic stability, to increase resistance to nuclease degradation (e.g., exonuclease resistant), and/or to increase binding specificity and minimize off-target effects. For example, thermodynamic stability can be determined based on measurement of melting temperature Tₘ. A higher Tₘ can be associated with a more thermodynamically stable chemical entity.

In some embodiments, the modification can render one or more of the nucleic acid strands in the nucleic acid complex to resist exonuclease degradation/cleavage. The term “exonuclease” as used herein, indicates a type of enzyme that works by cleaving nucleotides one at a time from the end (exo) of a polynucleotide chain. A hydrolyzing reaction that breaks phosphodiester bonds at either the 3′ or the 5′ end occurs. A 3′ and 5′ exonuclease can degrade RNA and DNA in cells, and can degrade RNA and DNA in the interstitial space between cells and in plasma, with a high efficiency and a fast kinetic rate. A close relative is the endonuclease, which cleaves phosphodiester bonds in the middle (endo) of a polynucleotide chain. 3′ and 5′ exonuclease and exonucleolytic complexes can degrade RNA and DNA in cells, and can degrade RNA and DNA in the interstitial space between cells and in plasma. The term “exoribonuclease” as used herein, refers to exonuclease ribonucleases, which are enzymes that degrade RNA by removing terminal nucleotides from either the 5′ end or the 3′ end of the RNA molecule. Enzymes that remove nucleotides from the 5′ end are called 5′-3′ exoribonucleases, and enzymes that remove nucleotides from the 3′ end are called 3′-5′ exoribonucleases.

The modification can comprise phosphonate modification, ribose modification (in the sugar portion), and/or base modification.

In some embodiments, the modified nucleotide can comprise modifications to the sugar portion of the nucleotides. For example, the 2′ OH-group of a nucleotide can be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. In some embodiments, the 2′ OH-group of a nucleotide or nucleoside is replaced by 2′ O-methyl group and the modified nucleotide or nucleoside is a 2′-O-methyl nucleotide or 2′-O-methyl nucleoside (2′-OMe). The 2′-O-methyl nucleotide or 2′-O-methyl nucleoside can be 2′-O-methyladenosine, 2′-O-methylguanosine, 2′-O-methyluridine, or 2′-O-methylcytidine. In some embodiments, the 2′ OH-group of a nucleotide is replaced by fluorine (F), and the modified nucleotide or nucleoside is a 2′-F nucleotide or 2′-F nucleoside (2′-deoxy-2′-fluoro or 2′-F). The 2′-F nucleotide or 2′-F nucleoside can be 2′-F-adenosine, 2′-F-guanosine, 2′-F-uridine, or 2′-F-cytidine. The modifications can also include other modifications such as nucleoside analog phosphoramidites. In some embodiments, glycol nucleic acids can be used.

In some embodiments, the modified nucleotide can comprise a modification in the phosphate group of the nucleotide, e.g., by substituting one or more of the oxygens of the phosphate group with sulfur or a methyl group. In some embodiments, one or more of the nonbridging oxygens of the phosphate group of a nucleotide is replaced by a sulfur.

In some embodiments, the nucleic acid strands herein described comprise one or more non-standard internucleoside linkage that is not a phosphodiester linkage. In some embodiments, the nucleic acid strands herein described comprise one or more phosphorothioate internucleoside linkages. The term “phosphorothioate linkage” (PS) as used herein, indicates a bond between nucleotides in which one of the nonbridging oxygens is replaced by a sulfur. In some embodiments, both nonbridging oxygens may be replaced by a sulfur (PS2). In some embodiments, one of the nonbridging oxygens may be replaced by a methyl group. The term “phosphodiester linkage” as described herein indicates the normal sugar phosphate backbone linkage in DNA and RNA wherein a phosphate bridges the two sugars. In some embodiments, the introduction of one or more phosphorothioate linkage in the core nucleic acid strand, the passenger nucleic acid strand, and/or the sensor nucleic acid strand can endow the modified nucleotides with increased resistance to nucleases (e.g., endonucleases and/or exonucleases).

In some embodiments, the modified nucleotide can comprise modifications to or substitution of the base portion of a nucleotide. For example, uridine and cytidine residues can be substituted with pseudouridine, 2-thiouridine, N6-methyladenosine, 5-methycytidine or other base analogs of uridine and cytidine residues. Adenosine can comprise modifications to Hoogsteen (e.g., 7-triazolo-8-aza-7-deazaadenosines) and/or Watson-Crick face of adenosine (e.g., N²-alkyl-2-aminopurines). Examples of adenosine analogs also include Hoogsteen or Watson-Crick face-localized N-ethylpiperidine triazole-modified adenosine analogs, N-ethylpiperidine 7-EAA triazole (e.g., 7-EAA, 7-ethynyl-8-aza-7-deazaadenosine) and other adenosine analogs identifiable to a person skilled in the art. Cytosine may be substituted with any suitable cytosine analogs identifiable to a person skilled in the art. For example, cytosine can be substituted with 6′-phenylpyrrolocytosine (PhpC) which has shown comparable base pairing fidelity, thermal stability and high fluorescence.

In some embodiments, one or more nucleotides in the nucleic acid complex disclosed herein can be substituted with a universal base. The term “universal base” refers to nucleotide analogs that form base pairs with each of the natural nucleotides with little discrimination between them. Examples of universal bases include, but are not limited to, hypoxanthine and derivatives thereof, inosine and derivatives thereof, azole carboxamide and derivatives thereof, nitroazole and derivatives thereof (e.g., 3-nitropyrrole, 4-nitroindole, 5-nitroindole, 6-nitroindole, nitroimidazole, and 4-nitropyrazole), phenyl C-ribonucleoside and derivatives thereof, naphthyl C-ribonucleoside and derivatives thereof, and other aromatic derivatives, or a combination thereof. In some embodiments, the universal bases comprised in the nucleic acid complex herein described comprise inosine or analogues thereof. Analogues of inosine include, for example, 2′-deoxyisoinosine, 7-deaza-2′-deoxyinosine, and 2-aza-2′-deoxyinosine. Examples of universal base and analogues thereof are described, for example, in Loakes, 2001, Nucleic Acids Research, 29, 2437-2447, the content of which is incorporated by reference in its entirety.

In some embodiments, base modification disclosed herein can reduce innate immune recognition while making the nucleic acid complex more resistant to nucleases. Examples of base modifications that can be used in the nucleic acid complex disclosed herein are also described, for example, in Hu et al. (Signal Transduction and targeted Therapy 5:101 (2020)), the content of which is incorporated by reference in its entirety. In some embodiments, the base modification disclosed herein (e.g., universal base) can result in reduced base pairing strength, thus decreasing the thermodynamics stability and the melting temperature of a formed duplex (e.g., sensor duplex).

In some embodiments, the nucleic acid strands (the core nucleic acid strand, the passenger nucleic acid strand, and/or the sensor nucleic acid strand) comprised in the nucleic acid complexes herein described can comprise one or more locked nucleic acids or analogs thereof. Exemplary locked nucleic acid analogs include, for example, their corresponding locked analog phosphoramidites and other derivatives apparent to a skilled artisan.

As used herein, the term “locked nucleic acids” (LNA) indicates a modified RNA nucleotide. The ribose moiety of an LNA nucleotide is modified with an extra bridge connecting the 2′ and 4′ carbons (a 2′-0, 4′-C methylene bridge). The bridge “locks” the ribose in the 3′-endo structural conformation and restricts the flexibility of the ribofuranose ring, thereby locking the structure into a rigid bicyclic formation. LNA nucleotides can be mixed with DNA or RNA bases in the oligonucleotide whenever desired. The incorporation of LNA into the nucleic acid complexes disclosed herein can increase the thermal stability (e.g., melting temperature), hybridization specificity of oligonucleotides as well as accuracies in allelic discrimination. LNA oligonucleotides display hybridization affinity toward complementary single-stranded RNA and complementary single-or double-stranded DNA. Additional information about LNA can be found, for example, at www.sigmaaldrich.com/technical-documents/articles/biology/locked-nucleic-acids-faq.html. In some embodiments, glycol nucleic acids can be used.

The nucleic acid strands (the core nucleic acid strand, the passenger nucleic acid strand, and/or the sensor nucleic acid strand) comprised in the nucleic acid complexes herein described can comprise other chemically modified nucleotide or nucleoside with 2′-4′ bridging modifications. A 2′-4′ bridging modification refers to the introduction of a bridge connecting the 2′ and 4′ carbons of a nucleotide. The bridge can be a 2′-0, 4′-C methylene bridge (e.g., in LNA). The bridge can also be a 2′-0, 4′-C ethylene bridge (e.g., in ethylen-bridged nucleic acids (ENA)) or any other chemical linkage identifiable to one of skill in the art.

The introduction of LNA, analogues thereof, or other chemically modified nucleotides with 2′-4′ bridging modifications in the nucleic acid complex herein described can enhance hybridization stability as well as mismatch discrimination. For example, a nucleic acid complex comprising a sensor nucleic acid strand with LNA, analogues thereof, or other chemically modified nucleotides with 2′-4′ bridging modifications can have an enhanced sensitivity to distinguish between matched and mismatched input nucleic acid strand (e.g., in the complementary binding between an input nucleic acid strand and a sensor nucleic acid strand).

In some embodiments, one or more of the nucleic acid strands of the nucleic acid complex can comprise a chemical moiety linked to the 3′ and/or 5′ terminus of the strand. The terminal moiety can include one or more any suitable terminal linkers or modifications. For example, the terminal moiety can include a linker to link the oligonucleotide with another molecule or a particular surface (biotins, amino-modifiers, alkynes, thiol modifiers, azide, N-Hydroxysuccinimide, and cholesterol), a dye (e.g., fluorophore or a dark quencher), a fluorine modified ribose, a space (e.g., C3 spacer, Spacer 9, Spacer 18, dSpacer, tri-ethylene glycol spacer, hexa-ethylene glycol spacer), moieties and chemical modification involved in click chemistry (e.g., alkyne and azide moieties), and any linkers or terminal modifications that can be used to attach the 3′ and 5′ end to other chemical moieties such as antibodies, gold or other metallic nanoparticles, polymeric nanoparticles, dendrimer nanoparticles, small molecules, single chain or branched fatty acids, peptides, proteins, aptamers, and other nucleic acid strands and nucleic acid nanostructures. The terminal moiety can serve as a label capable of detection or a blocker to protect a single-stranded nucleic acid from nuclease degradation. Additional linkers and terminal modification that can be attached to the terminus of the sensor nucleic acid strand are described in www.idtdna.com/pages/products/custom-dna-rna/oligo-modifications and www.glenresearch.com/browse/labels-and-modifiers, the contents of which are incorporated herein by reference in their entirety.

Additional modifications to the nucleotides and/or nucleosides can also be introduced to one or more strands of the nucleic acid complex herein described, such as modifications described in Hu et al. (Signal Transduction and targeted Therapy 5:101 (2020)), the content of which is incorporated by reference in its entirety.

### Ribose Modification

The percentage of the modified nucleosides of the nucleic acid complex can vary in different embodiments. In some embodiments, the percentage of the modified nucleosides of the nucleic acid complex herein described can be, be about, be at least, or be at least about 80%, 85%, 90%, or 95%. For example, percentage of the modified nucleosides of the nucleic acid complex herein described can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, at least 90%, 91%, 92%, 93%, 94%, 95%, or a number or a range between any two of these values of the nucleotides of the nucleic acid complex are modified (e.g., non-DNA and non-RNA). In some embodiments, all of the nucleotides of the nucleic acid complex are modified (e.g., non-DNA and non-RNA).

The percentage of the modified nucleosides in one or more strands of the nucleic acid complex can vary in different embodiments. The percentage of the modified nucleosides in a core nuclei acid strand herein described can be, be about, be at least, or be at least about 80%, 85%, 90%, or 95%. For example, the percentage of the modified nucleosides in a core nuclei acid strand herein described can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of a core nucleic acid strand are chemically modified.

In some embodiments, the percentage of the modified nucleosides in the region of a core nuclei acid strand complementarily bound to a passenger nucleic acid strand (e.g., the central region in Design 1 and Design 2 of FIGS. 1 and 3 or the first region in Design 3 of FIGS. 2-3) can be, be about, be at least, or be at least about 80%, 85%, 90%, or 95%. For example, the percentage of the modified nucleosides in the region of a core nuclei acid strand complementarily bound to a passenger nucleic acid strand can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the first region of a core nucleic acid strand are chemically modified.

The percentage of the modified nucleosides in the region of a core nucleic acid strand complementarily bound to a sensor nucleic acid strand can be, be about, be at least, or be at least about 80%, 85%, 90%, or 95%.

In some embodiments wherein a core nucleic acid strand comprises a 5′ region, a central region and a 3′ region, the percentage of the modified nucleosides in the 5′ region of a core nuclei acid strand herein described can be, be about, be at least, or be at least about 80%, 85%, 90%, or 95%. For example, the percentage of the modified nucleosides in the 5′ region of a core nuclei acid strand herein described can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the 5′ region of a core nucleic acid strand are chemically modified.

The percentage of the modified nucleosides in the 3′ region of a core nuclei acid strand herein described can be, be about, be at least, or be at least about 80%, 85%, 90%, or 95%. For example, the percentage of the modified nucleosides in the 3′ region of a core nuclei acid strand herein described can be, be about, be at least, or be at least about 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the 3′ region of a core nucleic acid strand are chemically modified.

In some embodiments wherein a core nucleic acid strand comprises a first region and a second region, the percentage of the modified nucleosides in the first region of a core nuclei acid strand herein described can be, be about, be at least, or be at least about 50%, 60%, 70%, 80%, 85%, 90%, or 95%. For example, the percentage of the modified nucleosides in the first region of a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the first region of a core nucleic acid strand are chemically modified.

In some embodiments, the percentage of the modified nucleosides in the second region of a core nuclei acid strand herein described can be, be about, be at least, or be at least about 50%, 60%, 70%, 80%, 85%, 90%, or 95%. For example, the percentage of the modified nucleosides in the second region of a core nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, all of the nucleosides of the second region of a core nucleic acid strand are chemically modified.

The percentage of the modified nucleosides in a passenger nuclei acid strand herein described can be, be about, be at least, or be at least about 50%, 60%, 70%, 80%, 85%, 90%, or 95%. For example, the percentage of the modified nucleosides in a passenger nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. In some embodiments, the percentage of the modified nucleosides in a passenger nuclei acid strand herein described can be, be about, be at least, or be at least about 80%, 85%, 90%, or 95%. In some embodiments, all of the nucleosides of a passenger nucleic acid strand are chemically modified.

The percentage of the modified nucleosides in a sensor nuclei acid strand herein described can be, be about, be at least, or be at least about 50%, 60%, 70%, 80%, 85%, 90%, or 95%. For example, the percentage of the modified nucleosides in a sensor nuclei acid strand herein described can be, be about, be at least, or be at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or a number or a range between any two of these values. The percentage of the modified nucleosides in a sensor nuclei acid strand herein described can be, be about, be at least, or be at least about 80%, 85%, 90%, or 95%. In some embodiments, all of the nucleosides of a sensor nucleic acid strand are chemically modified. The modified nucleosides in one or more of the core nucleic acid strand, the passenger nucleic acid strand, and the sensor nucleic acid strand can comprise 2′-O-methyl nucleoside and/or 2′-F nucleoside.

In some embodiments, the percentage of 2′-O-methyl nucleoside and/or 2′-F nucleoside in the nucleic acid complex herein described can be, be about, be at least, be at least about, be at most, or be at most about 10%-50%. For example, the percentage of 2′-O-methyl nucleoside and/or 2′-F nucleoside in the nucleic acid complex herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a core nucleic acid strand herein described can be, be about, be at least, be at least about, be at most, or be at most about 10%-50%. For example, the percentage of 2′-O-methyl nucleoside and/or 2′-F nucleoside in a core nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a passenger nucleic acid strand herein described can be, be about, be at least, be at least about, be at most, or be at most about 10%-50%. For example, the percentage of 2′-O-methyl nucleoside and/or 2′-F nucleoside in a passenger nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

In some embodiments, the percentage of 2′-O-methyl nucleoside and/or 2′-F nucleoside in a sensor nucleic acid strand herein described can be, be about, be at least, be at least about, be at most, or be at most about 10%-50%. For example, the percentage of 2′-O-methyl nucleoside and/or 2′-F nucleoside in a sensor nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

### Phosphate Modification

The percentage of phosphate modification to the nucleotides in the nucleic acid complex described herein can vary in different embodiments. In some embodiments, the phosphate modification comprises or is a phosphorothioate internucleoside linkage. In some embodiments, the percentage of phosphorothioate internucleoside linkages in a core nucleic acid strand is less than 5%, less than 10%, less than 25%, less than 50%, or a number or a range between any two of these values. For example, percentage of phosphorothioate internucleoside linkages in a core nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values. In some embodiments, the core nucleic acid strand does not comprise a phosphorothioate internucleoside linkage modification.

The percentage of phosphodiester internucleoside linkages in a core nucleic acid strand is about, at least, or at least about 50%, 80% or 95%, or a number or a range between any two of these values. For example, percentage of phosphodiester internucleoside linkages in a core nucleic acid strand is about, at least, or at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, all the internucleoside linkages in the core nucleic acid strand are phosphodiester internucleoside linkage.

In some embodiments wherein a core nucleic acid strand comprises a 5′ region, a central region, and a 3′ region (e.g., Design 2 in FIGS. 1 and 3), the 5′ terminus of the central region of the core nucleic acid strand comprises at least one phosphorothioate internucleoside linkage (e.g., one, two or three phosphorothioate internucleoside linkage). In some embodiments, the 3′ terminus of the central region of the core nucleic acid strand comprises at least one phosphorothioate internucleoside linkage (e.g., one, two or three phosphorothioate internucleoside linkage). In some embodiments, each of the 5′ terminus of the central region of the core nucleic acid strand and the 3′ terminus of the central region of the core nucleic acid strand independently comprises one or more phosphorothioate internucleoside linkages (e.g., one, two or three phosphorothioate internucleoside linkage). In some embodiments, the central region of the core nucleic acid strand does not comprise phosphorothioate internucleoside linkages except for the phosphorothioate internucleoside linkage(s) between two or three nucleosides at the 5′ terminus, 3′ terminus, or both, of the central region.

In some embodiments, the internucleoside linkages between the one to three nucleotides (e.g., one, two, or three nucleotides) adjacent to the 3′ of the 5′ connector of the core nucleic acid strand are phosphorothioate internucleoside linkages. In some embodiments, the internucleoside linkages between the one or two nucleotides adjacent to the 5′ of the 3′ connector of the core nucleic acid strand are phosphorothioate internucleoside linkages. In some embodiments, the internucleoside linkages between the one to three nucleotides (e.g., one, two, or three nucleotides) adjacent to the 3′ of the 3′ connector of the core nucleic acid strand are phosphorothioate internucleoside linkages. In some embodiments, the 3′ region of the core nucleic acid strand does not comprise phosphorothioate internucleoside linkages except for the phosphorothioate internucleoside linkage(s) between the one to three nucleotides (e.g., one, two, or three nucleotides) adjacent to the 3′ of the 3′ connector of the core nucleic acid strand. In some embodiments, the 5′ region of the core nucleic acid strand does not comprise phosphorothioate internucleoside linkages.

In some embodiments wherein a core nucleic acid strand comprises a first region and a second region (e.g., Design 3 in FIGS. 2-3), the 3′ terminus of the first region of the core nucleic acid strand comprises at least one phosphorothioate internucleoside linkage (e.g., one, two or three phosphorothioate internucleoside linkage). The phosphorothioate internucleoside linkage can be between the last two, three, or four nucleosides at the 3′ terminus of the first region of the core nucleic acid strand. In some embodiments, the 5′ terminus of the first region of the core nucleic acid strand comprises at least one phosphorothioate internucleoside linkage (e.g., one, two or three phosphorothioate internucleoside linkage). The phosphorothioate internucleoside linkage can be between the last two, three, or four nucleosides at the 5′ terminus of the first region of the core nucleic acid strand. In some embodiments, each of the 5′ terminus of the first region of the core nucleic acid strand and the 3′ terminus of the first region of the core nucleic acid strand independently comprises one or more phosphorothioate internucleoside linkages (e.g., one, two or three phosphorothioate internucleoside linkage). In some embodiments, the first region of the core nucleic acid strand does not comprise phosphorothioate internucleoside linkages except for the phosphorothioate internucleoside linkage(s) between the last two or three nucleosides at the 5′ terminus, 3′ terminus, or both, of the first region. For example, the first region of the core nucleic acid strand does not comprise phosphorothioate internucleoside linkages except for the phosphorothioate internucleoside linkage(s) between the last three nucleosides at the 5′ terminus and the last three nucleosides 3′ terminus of the first region.

In some embodiments, the percentage of phosphorothioate internucleoside linkages in the second region of a core nucleic acid strand is less than 5%, less than 10%, or a number or a range between any two of these values. In some embodiments, the second region of a core nucleic acid strand does not comprise phosphorothioate internucleoside linkages.

In some embodiments, the passenger nucleic acid strand comprises one or more phosphorothioate internucleoside linkage. The percentage of phosphorothioate internucleoside linkages in a passenger nucleic acid strand is less than 5%, less than 10%, less than 25%, less than 50%, or a number or a range between any two of these values. For example, percentage of phosphorothioate internucleoside linkages in a passenger nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

In some embodiments, the 5′ terminus of the passenger nucleic acid strand comprises at least one phosphorothioate internucleoside linkage (e.g., one, two, or three phosphorothioate internucleoside linkage). In some embodiments, the 3′ terminus of the passenger nucleic acid strand comprises at least one phosphorothioate internucleoside linkage (e.g., one, two, or three phosphorothioate internucleoside linkage). In some embodiments, the passenger nucleic acid strand does not comprise phosphorothioate internucleoside linkages except for the phosphorothioate internucleoside linkage(s) between the last two, three, or four nucleosides at the 5′ terminus, 3′ terminus, or both, of the passenger nucleic acid strand. In some embodiments, the passenger nucleic acid strand does not comprise phosphorothioate internucleoside linkages except for the phosphorothioate internucleoside linkage(s) between the last two to three nucleosides at the 5′ terminus and the last two to three nucleosides at 3′ terminus of the passenger nucleic acid strand.

The sensor nucleic acid strand can comprise one or more phosphorothioate internucleoside linkage. The percentage of phosphorothioate internucleoside linkages in a sensor nucleic acid strand can be less than 5%, less than 10%, less than 25%, less than 50%, less than 60%, less than 70% or a number or a range between any two of these values. For example, percentage of phosphorothioate internucleoside linkages in a sensor nucleic acid strand is about, less than, or less than about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, or a number or a range between any two of these values.

In some embodiments, the 5′ terminus of the sensor nucleic acid strand comprises at least one phosphorothioate internucleoside linkage (e.g., one, two or three phosphorothioate internucleoside linkage). In some embodiments, the 3′ terminus of the sensor nucleic acid strand comprises at least one phosphorothioate internucleoside linkage (e.g., one to twenty phosphorothioate internucleoside linkage. In some embodiments, each of the 5′ terminus of the sensor nucleic acid strand and the 3′ terminus of the sensor nucleic acid strand independently comprises one or more phosphorothioate internucleoside linkages (e.g., one, two or three at the 5′ terminus or one to twenty at the 3′ terminus). In some embodiments, the sensor nucleic acid strand does not comprise phosphorothioate internucleoside linkages except for the phosphorothioate internucleoside linkage(s) at the 5′ terminus, 3′ terminus, or both, of the sensor nucleic acid strand. In some embodiments, the phosphorothioate internucleoside linkages at the 3′ terminus of the sensor nucleic acid strand are in the singled-stranded overhang of the sensor nucleic acid strand.

### LNA, Analogues Thereof and 2′-4′Bridging Modification

The percentage of the LNA or analogues thereof of the nucleic acid complex can vary in different embodiments. In some embodiments, the percentage of the LNA or analogues thereof of the nucleic acid complex herein described can be about 10%-50%. For example, the percentage of the LNA or analogues thereof of the nucleic acid complex herein described can be about, at most, at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

The percentage of the LNA or analogues thereof in one or more strands of the nucleic acid complex can vary in different embodiments. In some embodiments, the percentage of the LNA or analogues thereof in a core nucleic acid strand herein described can be, be about, be at most, or be at most about 5%, 10%, or 15%. For example, the percentage of the LNA or analogues thereof of a core nucleic acid strand herein described can be, be about, be at most, or be at most about 5%, 6%, 7%, 8%, 9%, 10%, 11%, 12%, 13%, 14%, 15%, or a number or a range between any two of these values.

The percentage of the LNA or analogues thereof in a passenger nucleic acid strand herein described can be, be about, be at most, or be at most about 5%, 10%, or 15%. For example, the percentage of the LNA or analogues thereof of a passenger nucleic acid strand herein described can be, be about, be at most, or be at most about 5%, 6%, 7%, 8%, 9%, 10%, 11%, 12%, 13%, 14%, 15%, or a number or a range between any two of these values. In some embodiments, a percentage of the LNA or analogues thereof in a passenger nucleic acid strand herein described greater than 5%, greater than 10%, or greater than 15% can decrease the RNAi activity of the nucleic acid complex (see e.g., Example 1).

The percentage of the LNA or analogues thereof in a sensor nucleic acid strand herein described can be, be about, be at least, be at least about, be at most, or be at most about 10%-50%. For example, the percentage of the LNA or analogues thereof of a sensor nucleic acid strand herein described can be, be about, be at least, be at least about, be at most, or be at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or a number or a range between any two of these values.

The percentage of 2′-4′ bridging modification of the nucleic acid complex can vary in different embodiments. In some embodiments, the percentage of the 2′-4′ bridging modification of the nucleic acid complex herein described can be about 10%-50%. For example, the percentage of the 2′-4′ bridging modification of the nucleic acid complex herein described can be about, at most, at most about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or a number or a range between any two of these values.

### Base Modification

In some embodiments wherein a core nucleic acid strand comprises a 5′ region, a central region, and a 3′ region (e.g., Design 2 in FIGS. 1 and 3), the 5′ region and/or the 3′ region of the core nucleic acid strand can comprise one or more universal base herein described. For example, the 5′ region and/or the 3′ region of the core nucleic acid strand can comprise one or more inosine.

In some embodiments wherein a core nucleic acid strand comprises a first region and a second region (e.g., Design 3 in FIGS. 2-3), the second region of the core nucleic acid strand can comprise one or more universal base herein described. For example, the second region of the core nucleic acid strand can comprise one or more inosine.

In some embodiments, a sensor nucleic acid, and particularly the region of the sensor nucleic acid that is complementarily bound to a core nucleic acid strand, can comprise one or more universal base herein described.

### Core Strand

The core nucleic acid strand of the nucleic acid complex described herein can comprise a 5′ region, a 3′ region, and a central region between the 5′ region and the 3′ region.

Each of the 5′ region, the 3′ region, and the central region can be directly adjacent to each other, that is no nucleotide between the two adjacent regions. In some embodiments, the 3′ end of the 5′ region can be 1, 2, 3, 4, 5, 8, 9, 10, 11, 12, 13, 14, 15, 20, or a number or a range between any two of these values, nucleotides away from the 5′ end of the central region. In some embodiments, the 5′ end of the 3′ region can be 1, 2, 3, 4, 5, 8, 9, 10, 11, 12, 13, 14, 15, 20, or a number or a range between any two of these values, nucleotides away from 3′ of the central region. The core nucleic acid strand of the nucleic acid complex described herein can comprise a first region and a second region. The first region is at the 3′ direction of the second region.

The length of the core nucleic acid strand can vary in different embodiments. In some embodiments, the core nucleic acid strand comprises 20-70 linked nucleosides. For example, the core nucleic acid strand can comprise 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 linked nucleosides.

In some embodiments, the core nucleic acid strand comprises 20-60 linked nucleosides. For example, the core nucleic acid strand can comprise 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, linked nucleosides.

In some embodiments wherein a core nucleic acid strand comprises a 5′ region, a central region and a 3′ region (e.g., Design 2 in FIGS. 1 and 3), the length of the central region of the core nucleic acid strand can vary in different embodiments. In some embodiments, the central region of the core nucleic acid strand comprises 10-35 linked nucleosides. For example, the central region of the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides.

The 3′ region and the 5′ region of the core nucleic acid strand can have a same length or a different length. The length of the 3′ region and the 5′region of the core nucleic acid strand can vary in different embodiments. In some embodiments, the length of the 3′ region and the 5′region of the core nucleic acid strand comprises 2-33 linked nucleosides. For example, the 3′ region and the 5′region of the core nucleic acid strand can comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 linked nucleosides.

The central region of the core nucleic acid strand comprises a sequence complementary to a target RNA. The length of the sequence complementary to a target RNA can vary in different embodiments. In some embodiments, the sequence complementary to a target RNA is 10-21 nucleotides in length. For example, the sequence complementary to a target RNA is 10, 11, 12, 13, 14, 15, 16, 17, 18, 19, 20, or 21 nucleotides in length.

The central region of the core nucleic acid strand comprises a sequence complementary to a passenger nucleic acid strand. The length of the sequence complementary to a passenger nucleic acid strand can vary in different embodiments. In some embodiments, the sequence complementary to a passenger nucleic acid strand is 19-25 nucleotides in length. For example, the sequence complementary to a passenger nucleic acid strand is 19, 20, 21, 22, 23, 24, or 25 nucleotides in length.

In some embodiments wherein a core nucleic acid strand comprises a first region and a second region (e.g., Design 3 in FIGS. 2-3), the length of the first region of the core nucleic acid strand can vary in different embodiments. In some embodiments, the first region of the core nucleic acid strand comprises 10-30 linked nucleosides. For example, the first region of the core nucleic acid strand can comprise 10, 11, 12, 13, 14, 15, 16, 17, 18, 19, 20, 21, 22, 23, 24, 25, 26, 27, 28, 29, or 30, linked nucleosides. In some embodiments, the first region of the core nucleic acid strand comprises 17-22 linked nucleosides.

The length of the second region of the core nucleic acid strand can vary in different embodiments. In some embodiments, the length of the second region of the core nucleic acid strand comprises 10-30 linked nucleosides. For example, the second region of the core nucleic acid strand can comprise 10, 11, 12, 13, 14, 15, 16, 17, 18, 19, 20, 21, 22, 23, 24, 25, 26, 27, 28, 29, or 30, linked nucleosides. The first region and the second region of the core nucleic acid strand can have a same length or a different length. In some embodiments, the second region of the core nucleic acid strand has a relatively short length with respect to the first region of the core nucleic acid strand. In some embodiments, the second region of the core nucleic acid strand has about 14 linked nucleosides.

The first region of the core nucleic acid strand comprises a sequence complementary to a target RNA. The length of the sequence complementary to a target RNA can vary in different embodiments. In some embodiments, the sequence complementary to a target RNA is 10-35 nucleotides in length. For example, the sequence complementary to a target RNA is 10, 11, 12, 13, 14, 15, 16, 17, 18, 19, 20, 21, 22, 23, 24, 25, 26, 27, 28, 29, 30, 31, 32, 33, 34, or 35, nucleotides in length. In some embodiments, the sequence complementary to a target RNA is 10-21 nucleotides in length.

The first region of the core nucleic acid strand comprises a sequence complementary to a passenger nucleic acid strand. The length of the sequence complementary to a passenger nucleic acid strand can vary in different embodiments. In some embodiments, the sequence complementary to a passenger nucleic acid strand is 17-22 nucleotides in length. For example, the sequence complementary to a passenger nucleic acid strand is 17, 18, 19, 20, 21, or 22 nucleotides in length. In some embodiments, the sequence of the core nucleic acid strand complementary to a passenger nucleic acid strand is about 21 nucleotides in length.

In some embodiments, the regions of a core nucleic acid strand are connected to its adjacent regions via a connector. For example, in some embodiments wherein a core nucleic acid strand comprises a 5′ region, a central region, and a 3′ region (e.g., Design 2 in FIGS. 1 and 3), the central region of the core nucleic acid strand is linked to the 5′ region and the 3′ region of the core nucleic acid strand via a connector. For example, the central region of the core nucleic acid strand is linked the 5′ region of the core nucleic acid strand via a 5′ connector. In some embodiments, the central region of the core nucleic acid strand is linked to the 3′ region of the core nucleic acid strand via a 3′ connector.

In some embodiments wherein a core nucleic acid strand comprises a first region and a second region (e.g., Design 3 in FIGS. 2-3), the first region of the core nucleic acid strand is linked to the second region of the core nucleic acid strand via a connector. For example, the first region of the core nucleic acid strand is linked the second region of the core nucleic acid strand via a 5′ connector. In some embodiments, the core nucleic acid strand only comprises one connector (e.g., 5′ connector) and does not comprise a 3′ connector.

The 5′ connector and/or 3′ connector can comprise a three-carbon linker (C3linker), a nucleotide, any modified nucleotide described herein, or any moiety that can resist exonuclease cleavage when the core nucleic acid strand is single-stranded (e.g., after displacement of the sensor nucleic acid strand from the core nucleic acid strand). For example, the 5′ connector and/or the 3′ connector can comprise a 2′-F nucleotide such as 2′-F-adenosine, 2′-F-guanosine, 2′-F-uridine, or 2′-F-cytidine. The 5′ connector and/or the 3′ connector can comprise a 2′-O-methyl nucleotide such as 2′-O-methyladenosine, 2′-O-methylguanosine, 2′-O-methyluridine, or 2′-O-methylcytidine. The 5′ connector and/or the 3′ connector can comprise a naturally occurring nucleotide such as cytidine, uridine, adenosine, or guanosine. The 5′ connector and/or the 3′ connector of the core nucleic acid strand can comprise a phosphodiester linkage (phosphodiester 5′ and 3′ connection) cleavable by an exonuclease when in a single-stranded form. The 5′ connector and/or the 3′ connector of the core nucleic acid strand can comprise any suitable moiety that can resist exonuclease cleavage when in single-stranded form. In some embodiments, the 5′ connector of the core nucleic acid strand comprises no linker molecule except for the normal phosphodiester linkage connecting two adjacent nucleosides (see e.g., Design 3 in FIGS. 2-3).

In some embodiments, the 5′ connector can comprise or is, a C₃ 3-carbon linker, a nucleotide, a modified nucleotide (2′-O-methyl nucleotide, 2′-F nucleotide), a nucleotide with a phosphodiester 5′ and 3′ connection cleavable by an exonuclease when in a single stranded form, or a combination thereof. In some embodiments, the 5′ connector can comprise or is a 2′-O-methyl nucleotide such as 2′-O-methyladenosine, 2′-O-methylguanosine, 2′-O-methyluridine, or 2′-O-methylcytidine. In some embodiments, the 5′ connector can comprise or is 2′-F nucleotide such as 2′-F-adenosine, 2′-F-guanosine, 2′-F-uridine, or 2′-F-cytidine.

In some embodiments, the 3′ connector comprises or is, a C₃ 3-carbon linker, a nucleotide, a modified nucleotide, an exonuclease cleavage-resistant moiety when in a single stranded form, or a combination thereof. In some embodiments, the 3′ connector can comprise or is a 2′-O-methyl nucleotide such as 2′-O-methyladenosine, 2′-O-methylguanosine, 2′-O-methyluridine, or 2′-O-methylcytidine.

In some embodiments, the 3′ connector comprises or is a 2′-O-methyl nucleotide such as 2′-O-methyladenosine, 2′-O-methylguanosine, 2′-O-methyluridine, or 2′-O-methylcytidine and the 5′ connector comprises or is a 2′-O-methyl nucleotide such as 2′-0-methyladenosine, 2′-O-methylguanosine, 2′-O-methyluridine, or 2′-O-methylcytidine

In some embodiments, the 5′ connector of the core nucleic acid strand does not comprise or is not a C₃ 3-carbon linker. In some embodiments, the 3′ connector of the core nucleic acid strand comprises or is a C₃ 3-carbon linker. In some embodiments, it is advantageous to not have a C₃ 3-carbon linker as the 5′ connector. In some embodiments, it is advantageous to have a C₃ 3-carbon linker as the 3′ connector. In some embodiments, the 5′ connector of the core nucleic acid strand does not comprise or is not a C₃ 3-carbon linker, while the 3′ connector of the core nucleic acid strand comprises or is a C₃ 3-carbon linker.

In some embodiments, a nucleic acid complex not having a C₃ 3-carbon linker as the 5′ connector exhibit higher RNA interfering activity (see Examples 1-2). The nucleic acid complex can have a modified nucleotide or a nucleotide as the 5′ connector. The nucleic acid complex can have no 5′ connector. The nucleic acid complex can have a C₃ 3-carbon linker, a modified nucleotide, or a nucleotide as the 3′ connector. The nucleic acid complex can have no 3′ connector. In some embodiments, not having a C₃ 3-carbon linker as the 5′ connector increases RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these value, greater than nucleic acid complexes having a C₃ 3-carbon linker as the 5′ connector.

In some embodiments, a nucleic acid complex having a C₃ 3-carbon linker as the 3′ connector exhibit higher RNA interfering activity (see Examples 1-2). The nucleic acid complex can have a modified nucleotide or a nucleotide as the 5′ connector. The nucleic acid complex can have no 5′ connector. The nucleic acid complex does not have a C₃ 3-carbon linker as the 5′ connector. In some embodiments, having a C₃ 3-carbon linker as the 3′ connector increases RNA interfering activity of the nucleic acid complex by at least about 2-fold, 3-fold, 4-fold, 5-fold, 6-fold, 7-fold, 8-fold, 9-fold, 10-fold, 15-fold, 20-fold, or a number or a range between any of these value, greater than nucleic acid complexes having a modified nucleotide (e.g., 2′-O-methyl nucleotide) as the 3′ connector. In some embodiments, having a C₃ 3-carbon linker as the 3′ connector increases RNA interfering activity of the nucleic acid complex by at least about 2-fold, 3-fold, 4-fold, 5-fold, 6-fold, 7-fold, 8-fold, 9-fold, 10-fold, 15-fold, 20-fold, or a number or a range between any of these value, greater than nucleic acid complexes having no 3′ connector.

In some embodiments, the core nucleic acid strand do not comprise a 5′ connector and/or a 3′ connector. Instead, different regions of the core nucleic acid strand are linked to their adjacent regions via a standard phosphodiester linkage.

In some embodiments wherein a core nucleic acid strand comprises a 5′ region, a central region and a 3′ region (e.g., Design 2 in FIGS. 1 and 3), the central region of the core nucleic acid strand is linked the 3′ region and/or the 5′ region via a standard phosphodiester linkage. In some embodiments, the central region of the core nucleic acid strand is linked to the 5′ region of the core nucleic acid strand via a phosphodiester linkage. In some embodiments, the central region of the core nucleic acid strand is linked to the 3′ region of the core nucleic acid strand via a phosphodiester linkage. In some embodiments, the central region of the core nucleic acid strand is linked to the 3′ region of the core nucleic acid strand via a phosphodiester linkage, while the central region of the core nucleic acid strand is linked to the 5′ region of the core nucleic acid strand via a 2′-O-methyl nucleotide such as 2′-0-methyladenosine, 2′-O-methylguanosine, 2′-O-methyluridine, or 2′-O-methylcytidine. In some embodiments, the central region of the core nucleic acid strand is linked to the 5′ region of the core nucleic acid strand via a phosphodiester linkage, while the central region of the core nucleic acid strand is linked to the 3′ region of the core nucleic acid strand via a 2′-O-methyl nucleotide such as 2′-O-methyladenosine, 2′-O-methylguanosine, 2′-O-methyluridine, or 2′-O-methylcytidine. In some embodiments, the central region of the core nucleic acid strand is linked to the 3′ region and the 5′ region of the core nucleic acid strand both via a phosphodiester linkage.

In some embodiments wherein a core nucleic acid strand comprises a first region and a second region (e.g., Design 3 in FIGS. 2-3), the first region of the core nucleic acid strand is linked to the second region via a standard phosphodiester linkage connecting two adjacent nucleosides.

In some embodiments, not having a 5′ connector and/or a 3′ connector increases RNA interfering activity of the nucleic acid complex by at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold or a number or a range between any of these value, greater than nucleic acid complexes having a C₃ 3-carbon linker as the 5′ connector.

In some embodiments, a core nucleic acid strand can have an overhang. The overhang can be at the 3′ terminus of the core nucleic acid strand (3′ overhang). In some embodiments, the core nucleic acid strand can have a short overhang at the 3′ terminus (e.g., 1-3nucleosides), but the 3′ overhang does not extend back into the middle of the sensor duplex to bind with the sensor nucleic acid strand (see e.g., Design 3 in FIGS. 2-3). The length of the overhang can vary in different embodiments. In some embodiments, the 3′ overhang is about one to three nucleotides in length. For example, the 3′ overhang can be one, two or three nucleotides in length. The overhang can comprise one or more modified nucleotides, such as 2′-O-methyl nucleotides. For example, the 3′ overhang can comprise two 2′-O-methyl nucleotides (see e.g., Design 3 in FIGS. 2-3). The overhang can comprise modified internucleoside linkages, such as phosphorothioate internucleoside linkages. In some embodiments, all of the nucleotides in the overhang are chemically modified. In some embodiments, all of internucleoside linkages in the 3′ overhang of the core nucleic acid strand are phosphorothioate internucleoside linkages.

### Passenger Nucleic Acid Strand

The passenger nucleic acid strand of the nucleic acid complex described herein is complementary bound to a region of the core nucleic acid strand to form a RNAi duplex (e.g., a first nucleic acid duplex). In some embodiments, the core nucleic acid strand comprises a sequence complementary to a target nucleic acid strand. In some embodiments, the passenger nucleic strand of the nucleic acid complex can comprise a sequence homologous to the target nuclei acid strand.

As used herein, the term “homologous” or “homology” refers to sequence identity between at least two sequences. The term “sequence identity” or “identity” in the context of two nucleic acid or polypeptide sequences makes reference to the nucleotide bases or residues in the two sequences that are the same when aligned for maximum correspondence over a specified comparison window.

In some embodiments, the sequence identity between a passenger nucleic acid strand and a target nucleic acid or a portion there of can be, be about, be at least, or be at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values. The passenger nucleic acid strand of a nucleic acid complex can have a sequence substantially identical, e.g., at least 80%, 90%, or 100%, to a target nucleic acid or a portion thereof.

The length of the passenger nucleic acid strand can vary in different embodiments. In some embodiments, the passenger nucleic acid strand comprises 10-35 linked nucleosides. For example, the core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the passenger nucleic acid strand comprises 17-21 linked nucleosides.

The passenger nucleic acid strand can have a 3′ overhang, a 5′ overhang, or both in the RNAi duplex. In some embodiments, the passenger nucleic acid strand has a 3′ overhang, and the 3′ overhang is one to five nucleosides in length. In some embodiments, the overhang of the passenger nucleic acid strand is capable of binding to the input nucleic acid strand to form a toehold, thereby initiating a toehold mediated strand displacement and causing the displacement of the passenger nucleic acid strand from the core nucleic acid strand. In some embodiments, the overhang of the passenger nucleic acid strand is 5 to 20 nucleosides in length. For example, the overhang of the passenger nucleic acid strand can be 5, 6, 7, 8, 9, 10, 11, 12, 13, 14, 15, 16, 17, 18, 19, or 20 nucleosides in length. In some embodiments, the overhang of the passenger nucleic acid strand is 9 nucleosides in length.

In some embodiments, one or more internucleoside linages of the overhang of the passenger nucleic acid strand are phosphorothioate internucleoside linkage which can protect the overhang from degradation. In some embodiments, all internucleoside linages of the overhang of the passenger nucleic acid strand can be phosphorothioate internucleoside linkage.

In some embodiments, the passenger nucleic acid strand is fully complementary o the first region of the core nucleic acid strand, thereby forming no overhang at the 5′ and 3′ termini of the passenger nucleic acid strand in the RNAi duplex. Therefore, in some embodiments, the passenger nucleic acid strand does not have a 3′ overhang, a 5′ overhang, or both in the RNAi duplex. In some embodiments, having a blunt end with no overhang can render the passenger nucleic acid strand unfavorable for Dicer binding, thereby bypassing the Dicer-mediated cleavage.

In some embodiments, the passenger nucleic acid strand is attached to a terminal moiety and/or a blocking moiety. Any suitable terminal moiety described herein that is capable of blocking the passenger nucleic acid strand from interacting with a RNAi pathway enzyme (e.g., Dicer, RISC) can be used. The blocking moiety can include one or more suitable terminal linkers or modifications such as a blocker that can protect a single-stranded nucleic acid from nuclease degradation such as an exonuclease blocking moiety. Examples of suitable blocking moieties include, but are not limited to, a dye (e.g., fluorophore, Cy3, a dark quencher), inverted dT, a linker to link the oligonucleotide with another molecule or a particular surface (biotins, amino-modifiers, alkynes, thiol modifiers, azide, N-Hydroxysuccinimide, and cholesterol), a space (e.g., C3 spacer, Spacer 9, Spacer 18, dSpacer, tri-ethylene glycol spacer, hexa-ethylene glycol spacer), a fatty acid, one or more modified nucleotides (e.g., 2′-O-methyl, 2′-F, PS backbone connection, LNA, and/or 2′-4′ bridged base) or a combination thereof. In some embodiments, the 5′ terminus of the passenger nucleic acid is attached to an inverted-dT, a tri-ethylene-glycol, or a fluorophore. For example, a fluorophore can be attached to the 5′ terminus of the passenger nucleic acid strand via a phosphorothioate linkage.

### Sensor Nucleic Acid Strand

The sensor nucleic acid strand of the nucleic acid complex described herein comprises a region complementarily bound to at least a region of the core nucleic acid strand to form a sensor duplex (e.g., a second nucleic acid duplex). For example, the sensor nucleic acid strand can comprise a region complementarily bound to the 5′ region and the 3′ region of a core nucleic acid strand (e.g., in Design 2 of FIGS. 1 and 3). In another example, the sensor nucleic acid strand can comprise a region complementarily bound to the second region of a core nucleic acid strand (e.g., in Design 3 of FIGS. 2-3).

The length of the region complementarily bound to a core nucleic acid strand can vary in different embodiments. In some embodiments, the region complementarily bound to a core nucleic acid strand comprises 10-35 linked nucleosides. For example, the region in the sensor nucleic strand complementarily bound to a core nucleic acid strand can comprise 10, 11, 12, 13, 14, 15, 16, 17, 18, 19, 20, 21, 22, 23, 24, 25, 26, 27, 28, 29, 30, 31, 32, 33, 34, or 35 linked nucleosides. In some embodiments, the region complementarily bound to a core nucleic acid strand comprises 10-30 linked nucleosides. In some embodiments, the region in the sensor nucleic acid strand complementarily bound to a core nucleic acid strand comprise about 14linked nucleosides.

The sensor nucleic acid strand can comprise a toehold (overhang). The overhang can be at the 3′ end or 5′ end, or both, of the sensor nucleic acid strand. For example, the overhang can be at the 3′ of the sensor region complementary to the core nucleic acid strand. The overhang is not complementary to the core nucleic acid strand and is capable of binding to an input nucleic acid strand, thereby initiating a toehold mediated strand displacement and causing the displacement of the passenger nucleic acid strand from the core nucleic acid strand.

The length of the overhang in the sensor nucleic acid strand can vary in different embodiments. In some embodiments, the length of the overhang can be 5-20 linked nucleotides. For example, the length of the overhang in the sensor nucleic acid strand can comprise 5, 6, 7, 8, 9, 10, 11, 12, 13, 14, 15, 16, 17, 18, 19, or 20 nucleotides in length. In some embodiments, the overhang of the sensor nucleic acid strand is 12 nucleotides in length.

The overhang of the sensor nucleic acid strand can comprise nucleotide modification introduced to improve the base-pairing affinity, nuclease resistance of the singled-stranded overhang, and thermodynamic stability to avoid spurious exonuclease induced activation of the strand. Exemplary modifications include, but not limited to, 2′-O-methyl modification, 2′-Fluoro modifications, phosphorothioate internucleoside linkages, inclusions of LNA, and the like that are identifiable by a skilled person. In some embodiments, at least 50% of the internucleoside linkages in the overhang of the sensor nucleic acid strand are phosphorothioate internucleoside linkages. For example, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two values, of the internucleoside linkages in the overhang of the sensor nucleic acid strand are phosphorothioate internucleoside linkages. In some embodiments, all internucleoside linkages in the overhang of the sensor nucleic acid strand are phosphorothioate internucleoside linkages.

In some embodiments, the 5′ terminus and/or the 3′ terminus of the sensor nucleic acid strand can comprise a terminal moiety. Any suitable terminal moiety described herein can be used. In some embodiments, the terminal moiety can include a tri-or hexa-ethylene glycol spacer, a C3 spacer, an inverted dT, an amine linker, a ligand (e.g., a targeting ligand), a fluorophore, an exonuclease, a fatty acid, a Cy3, an inverted dT attached to a tri-ethylene glycol, or a combination thereof. In some embodiments, the 3′ terminus of the sensor nucleic acid strand can be attached to a delivery ligand, a dye (e.g., fluorophore), or exonuclease. The 5′ terminus can be attached to a fatty acid, a dye (e.g., Cy3), an inverted dT, a tri-ethylene glycol, or an inverted dT attached to a tri-ethylene glycol. The delivery ligand attached to the 3′ terminus can be any suitable ligand for use in targeting the nucleic acid complex to specific cell types described elsewhere in the present disclosure.

The sequence of the sensor nucleic acid strand can be designed to sense an input nucleic acid strand or a portion thereof. For example, from the sequence of an input biomarker, a list of all possible sensor segments which are antisense to the input strand can be generated. The sensor sequences for uniqueness in the transcriptome of the target animal can be ranked using NCBI BLAST. For human cancer cell lines, sequences can be checked against human transcript and genomic collection using the BLASTn algorithm. In some embodiments, sensor segments that have more than 17 bases of sequence complementarity and complete overhang complementarity to known or predicted RNA transcripts may be eliminated. Examples of design features of the sensor nucleic acid strand that can be used in the nucleic acid complexes described herein are described, for example, in the internatioanl patent application published as WO/2020/033938, the content of which is incorporated herein by reference.

### Input Nucleic Acid Strand

The input nucleic acid strand described herein acts as a trigger to activate (switch on) the RNA interfering activity of the nucleic acid complex (e.g., RNAi duplex) upon binding to a sequence of the sensor nucleic acid in the nucleic acid complex.

The input nucleic acid strand comprises a sequence complementary to a sequence in the sensor nucleic acid of the nucleic acid complex. The complementary binding between the input nucleic acid strand and the sensor nucleic acid strand (e.g., an overhang) causes displacement of the sensor nucleic acid strand from the core nucleic acid strand, thereby activating the RNA interfering activity of the RNAi duplex formed by the passenger nucleic acid strand and the central region of the core nucleic acid strand.

The input nucleic acid strand can be cellular RNA transcripts that are present at relatively high expression levels in a set of target cells (e.g., cancer cells) and at a relatively low level of expression in a set of non-target cells (e.g., normal cells). In some embodiments, the nucleic acid complex herein described is activated (switched on) in target cells. While in the non-target cells, the nucleic acid complex remains inactivated (switched off).

In the target cells, the input nucleic acid strand can be expressed at a level of, about, at least, or at least about 2-fold, 5-fold, 10-fold, 20-fold, 30-fold, 40-fold, 50-fold, 60-fold, 70-fold, 80-fold, 90-fold, or 100-fold higher than in the non-target cells.

In the target cells, the input nucleic acid strand can be expressed at a level of, about, at least, at least about 50, 100, 200, 300, 400, 500, 600, 700, 800, 900, 1000 transcripts. In some embodiments, in the non-target cells, the input nucleic acid strand is expressed at a level of less than 50, less than 40, less than 30, less than 20, or less than 10 transcripts. Preferably, the non-target cells have no detectable expression of the input nucleic acid strand.

The input nucleic acid strand can comprise an mRNA, an miRNA, or a non-coding RNA such as a long non-coding RNA, an RNA fragment, or an RNA transcript of a virus. In some embodiments, the input nucleic acid strand is an RNA transcript that is expressed in a set of cells that are causing the progression of a disease and are therefore targeted for RNAi therapy. The non-target cells are usually a set of cells where silencing of a target RNA can cause side effects that are not beneficial for therapy. For treating a disease or a condition where the input RNA is overexpressed in target cells, the nucleic acid complex can be designed such that the sensor nucleic acid strand comprises a sequence complementary to the input RNA sequence. Upon administration of the nucleic acid complex, the binding of sensor nucleic acid strand to the input RNA induces the dissociation of the RNAi duplex from the sensor duplex in target cells thereby to activate the RNAi targeting the disease or condition.

In some embodiments, the input nucleic acid strand comprises a biomarker. The term “biomarker” refers to a nucleic acid sequence (DNA or RNA) that is an indicator of a disease or disorder, a susceptibility to a disease or disorder, and/or of response to therapeutic or other intervention. A biomarker can reflect an expression, function or regulation of a gene. The input nucleic acid strand can comprise any disease biomarker known in the art.

In some embodiments, the input nucleic acid strand is a mRNA, for example a cell type or cell state specific mRNA. Examples of a cell type or cell-state specific mRNA include, but are not limited to, C3, GFAP, NPPA, CSFIR, SLC1A2, PLP1, and MBP mRNA. In some embodiments, the input nucleic acid is a microRNA (also known as miRNA), including but is not limited to, hsa-mir-23a-3p, hsa-mir-124-3p, and hsa-mir-29b-3p. In some embodiments, the input nucleic acid strand is a non-coding RNA, for example MALATI (metastasis associated lung adenocarcinoma transcript 1, also known as NEAT2 (noncoding nuclear-enriched abundant transcript 2).

### Target RNA

The core nucleic acid strand comprises a sequence complementary to a target RNA in order to direct target-specific RNA interference. In some embodiments, the target RNA is a cellular RNA transcript. The target RNA can be an mRNA, an miRNA, a non-coding RNA, a viral RNA transcript, or a combination thereof.

As used herein, a “target RNA” refers to a RNA whose expression is to be selectively inhibited or silenced through RNA interference. A target RNA can be a target gene comprising any cellular gene or gene fragment whose expression or activity is associated with a disease, a disorder or a condition. A target RNA can also be a foreign or exogenous RNA or RNA fragment whose expression or activity is associated with a disease, a disorder or a certain condition (e.g., a viral RNA transcript or a pro-viral gene).

The target RNA can comprise an oncogene, a cytokinin gene, an idiotype protein gene (Id protein gene), a prion gene, a gene that expresses a protein that induces angiogenesis, an adhesion molecule, a cell surface receptor, a gene of a protein involved in a metastasizing and/or invasive process, a gene of a proteinase, a gene of a protein that regulates apoptosis and the cell cycle, a gene that expresses the EGF receptor, a multi-drug resistance 1 gene (MDR1), a gene of a human papilloma virus, a hepatitis C virus, or a human immunodeficiency virus, a gene involved in cardiac hypertrophy, or a fragment thereof.

The target RNA can comprise a gene encoding for a protein involved in apoptosis. Exemplary target RNA genes include, but are not limited to, bcl-2, p53, caspases, cytotoxic cytokines such as TNF-α or Fas ligand, and a number of other genes known in the art as capable of mediating apoptosis. The target RNA can comprise a gene involved in cell growth. Exemplary target RNA genes include, but not limited to, oncogenes (e.g., genes encoding for ABLI, BCLI, BCL2, BCL6, CBFA2, CBL, CSFIR, ERBA, ERBB, EBRB2, ETSI, ETSI, ETV6, FGR, FOS, FYN, HCR, HRAS, JUN, KRAS, LCK, LYN, MDM2, MLL, MYB, MYC, MYCLI, MYCN, NRAS, PIM I, PML, RET, SRC, TALI, TCL3, and YES), as well as genes encoding for tumor suppressor proteins (e.g., APC, BRCA1, BRCA2, MADH4, MCC, NF I, NF2, RB I, TP53, and WTI). The target RNA can comprise a human major histocompatibility complex (MHC) gene or a fragment thereof. Exemplary MHC genes include MHC class I genes such as genes in the HLA-A, HLA-B or HLA-C subregions for class I cc chain genes, or β₂-microglobulinand and MHC class II genes such as any of the genes of the DP, DQ and DR subregions of class II α chain and β chain genes (i.e. DPα, DPβ, DQα, DQβ, DRα, and DRβ).

The target RNA can comprise a gene encoding for a pathogen-associated protein. Pathogen associated protein include, but are not limited to, a viral protein involved in immunosuppression of the host, replication of the pathogen, transmission of the pathogen, or maintenance of the infection, or a host protein which facilitates entry of the pathogen into the host, drug metabolism by the pathogen or host, replication or integration of the pathogen's genome, establishment or spread of infection in the host, or assembly of the next generation of pathogen. In some embodiments, the pathogen can be a virus, such as a herpesvirus (e.g., herpes simplex, varicella-zoster virus, Epstein-Barr virus, cytomegalovirus (CMV)), hepatitis C, HIV, JC virus), a bacteria or a yeast.

The target RNA can comprise a gene associated with a disease or a condition of the central nervous system (CNS). Exemplary genes associated with a CNS disease or a condition include, but are not limited to, APP, MAPT, SODI, BACEI, CASP3, TGM2, NFE2L3, TARDBP, ADRB1, CAMK2A, CBLNI, CDK5R1, GABRAI, MAPK10, NOSI, NPTX2, NRGN, NTS, PDCD2, PDE4D, PENK, SYTI, TTR, FUS, LRDD, CYBA, ATF3, ATF6, CASP2, CASPI, CASP7, CASP8, CASP9, HRK, CIQBP, BNIP3, MAPK8, MAPK14, Rac1, GSK3B, P2RX7, TRPM2, PARG, CD38, STEAP4, BMP2, GJAI, TYROBP, CTGF, ANXA2, RHOA, DUOX1, RTP801, RTP801L, NOX4, NOXI, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAPI, SHC1, ZNHIT1, LGALS3, HI95, SOX9, ASPP1, ASPP2, CTSD, CAPNS1, FAS and FASLG, NOGO and NOGO-R; TLR1, TLR2, TLR3, TLR4, TLR6, TLR7, TLR8, TLR9, IL1bR, MYD88, TICAM, TIRAP, and HSP47.

### EXAMPLES

Some aspects of the embodiments discussed above are disclosed in further detail in the following examples, which are not in any way intended to limit the scope of the present disclosure.

### Example 1

### RNAi Activity With or Without a C3 Linker

This example demonstrates the RNAi activity of various siRNA domain variants with or without a C3 linker as the 5′ and the 3′ connector.

The passenger and core strands of the new construct are assembled to form the siRNA domains of the new construct. The different variants of these siRNA domains are tested for RNAi activity.

To test the constructs, CASi siRNA segments were assembled by thermally annealing passenger and core strands in 1x phosphate buffer saline. The RNAi activities of the CASi siRNA segments were measured using dual luciferase assays. CASi siRNA segments were co-transfected into HCT 116 cells with dual luciferase vectors carrying the Huntingtin gene siRNA target sequence, using lipofectamine 2000. After 48 hours, cells were lysed and assayed for knockdown of the target gene by comparing the luminescence value of Renilla luciferase that carries the target sequence to Firefly luciferase that was used as a reference control. Methods and procedures of assembling CASi siRNA, cell transfection, and dual luciferase assays can be found in, for example, international application WO 2020/033938, the content of which is incorporated herein by reference in its entirety.

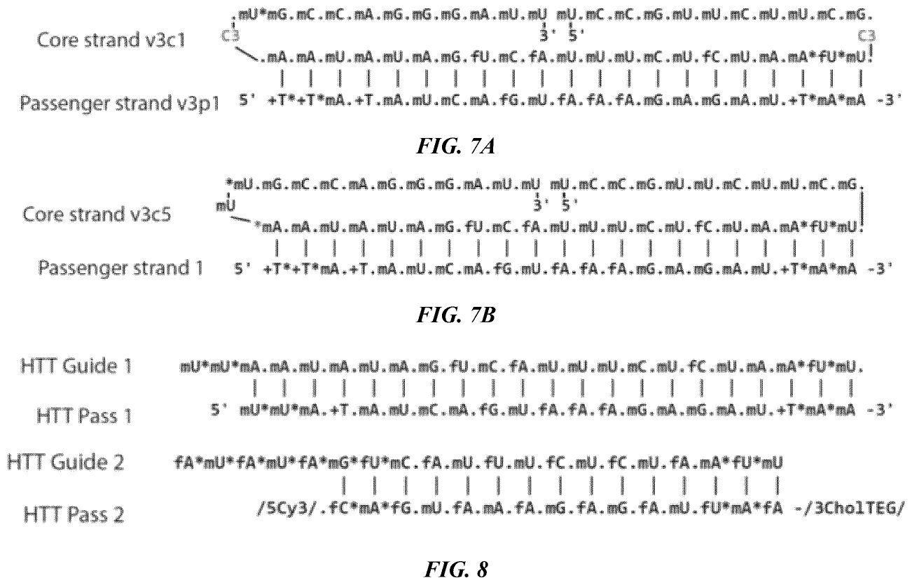

FIG. 7A and FIG. 7B show sequence diagrams of two exemplary nucleic acid complex constructs whose RNAi activities are determined in this example. Top nucleic acid complex construct comprises a core strand v3c1 base-paired to a passenger strand v3p1, in which a C3 linker is used as the 5′ and the 3′ connector. Bottom nucleic acid complex construct comprises a core strand v3c5 base-paired to the same passenger strand, in which no C3 linker is used as the 5′ and the 3′ connector. Instead, v3c5 core strand has a 3′ mU connector and no connector at the 5′ end.

FIG. 8 show sequence diagrams of two positive control nucleic acid complex constructs used in the assay described in this example.

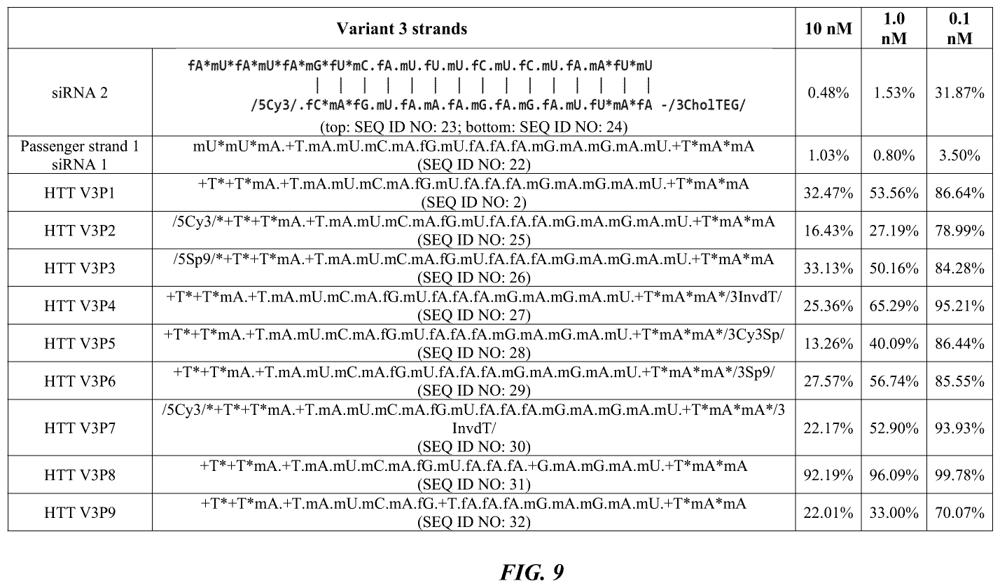

FIG. 9 shows various siRNA variants with different passenger strand (V3P1, V3P2, V3P3, V3P5, V3P5, V3P6, V3P7, V3P8, and V3P9) assembled with v3c1 core strand shown in FIG. 7A and tested in this example. The v3c1 core strand has a C3 linker as the 5′ and the 3′ connector. The target protein expression was tested with the siRNA variants at three different concentrations: 10 nM, 1.0 nM, and 0.1 nM.

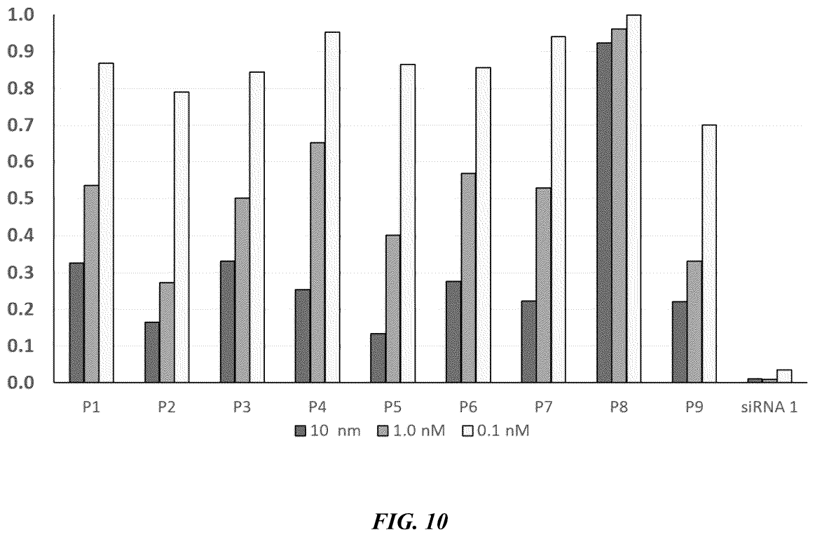

FIG. 10 shows a graphic representation of the target protein expression data for the siRNA variants shown in FIG. 9. Higher RNAi activity is suggested by lower expression of the target protein.

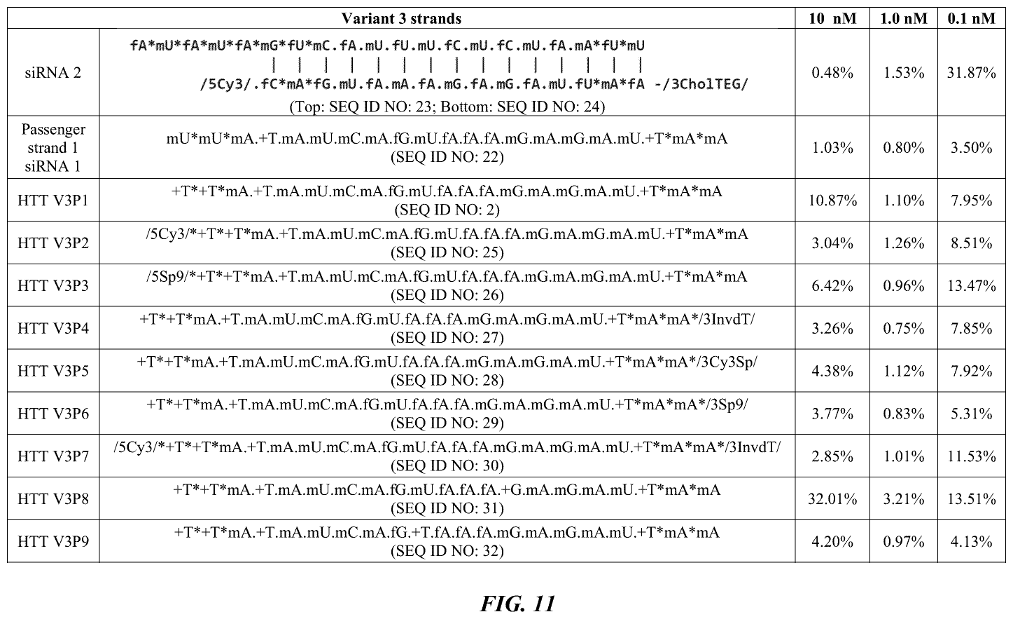

FIG. 11 shows different siRNA variants with different passenger strand (V3P1, V3P2, V3P3, V3P5, V3P5, V3P6, V3P7, V3P8, and V3P9) assembled with a v3c5 core strand shown in FIG. 7B and tested in this example. The v3c5 core strand does not have a C3linker as the 5′ and the 3′ connector. Instead, v3c5 core strand has a 3′ mU connector and no connector at the 5′ end. The target protein expression was tested with the siRNA variants at three different concentrations: 10 nM, 1.0 nM, and 0.1 nM.

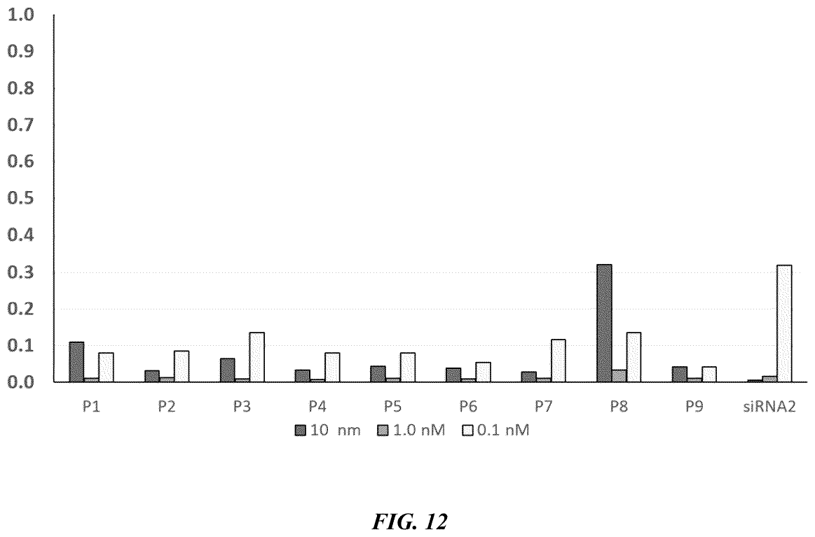

FIG. 12 shows a graphic representation of the target protein expression data for the siRNA variants shown in FIG. 11. Similar to FIGS. 9-10, higher RNAi activity is suggested by lower expression of the target protein.

These data indicate that a C3 linker as the 5′ connector inhibits RNAi activity of the siRNA domain. A comparison of the target protein expression data among different passenger variants (V3P1, V3P2, V3P3, V3P5, V3P5, V3P6, V3P7, V3P8, and V3P9) indicates that extensive modification of the passenger strand with LNAs (e.g., HTT V3P8) can decrease RNAi activity.

### Example 2

### RNAi Activity with Different 5′ and 3′ Connectors

In this example, different versions of the core strand were tested with the same sensor (Mir23 Sensor 1) and passenger strands (Passenger strand 1) to investigate the effects of different 5′ and 3′ connectors on the RNAi activity. RNAi activity was also evaluated between two-stranded constructs and three-stranded constructs.

Two-stranded constructs consist of the passenger strand base-paired to the core strand, forming an active siRNA domain. Three-stranded constructs consist of all three strands: the passenger strand, the core strand, and the sensor strand.

CASi siRNA segments (two-stranded constructs) and three-stranded constructs were assembled by thermally annealing passenger and core strands, or passenger, core and sensor strands in 1x phosphate buffer saline.

CASi siRNA segments or three-stranded constructs were co-transfected into HCT 116 cells with dual luciferase vectors carrying the Huntingtin gene siRNA target sequence, using lipofectamine 2000. After 48 hours, cells were lysed and assayed for knockdown of the target gene by comparing the luminescence value of Renilla luciferase that carries the target sequence to Firefly luciferase that was used as a reference control. Examples of methods and procedures of assembling CASi siRNA, cell transfection, and dual luciferase assays are described in, for example, international application WO 2020/033938.

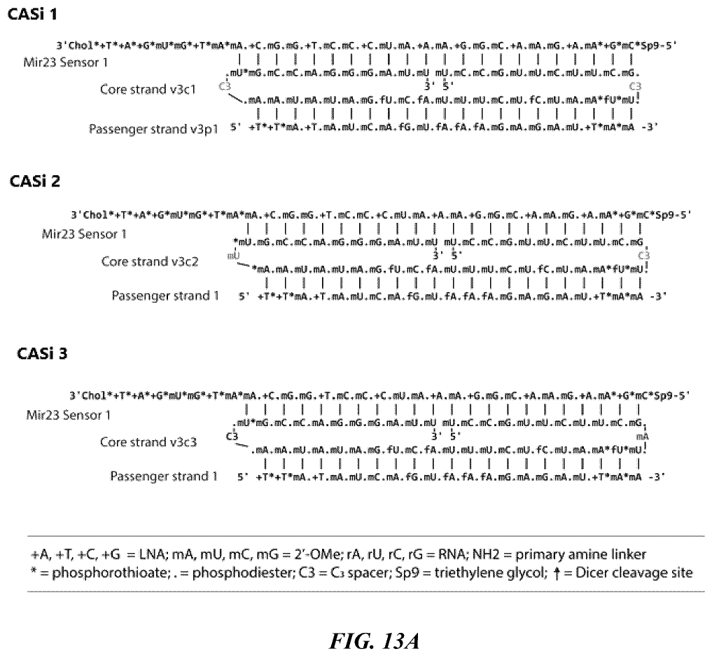

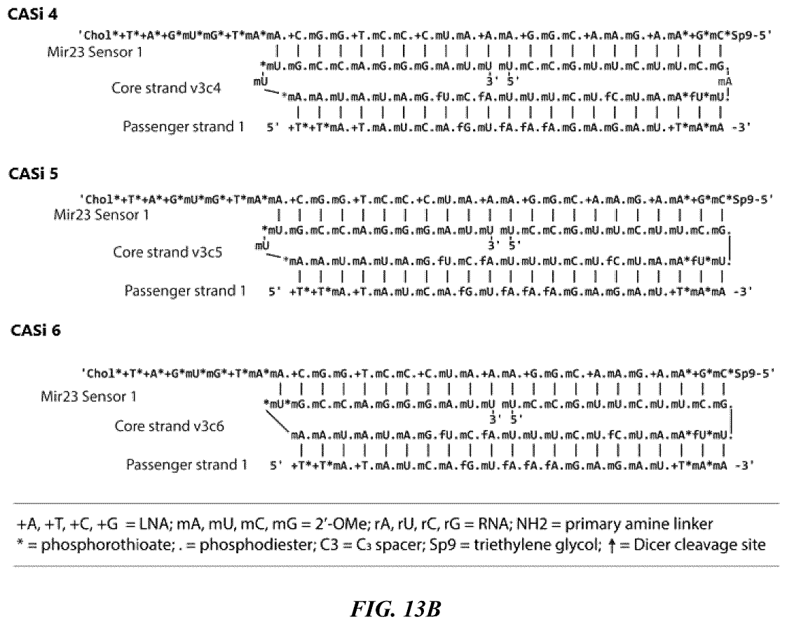

FIG. 13A and FIG. 13B shows sequence diagrams of various nucleic acid complexes disclosed herein each having the same passenger strand (Passenger strand 1) and the sensor strand (Mir23 Sensor 1) but a different core strand (Core strand v3c1, Core strand v3c2, Core strand v3c3, Core strand v3c4, Core strand v3c5, and Core strand v3c6), and particularly, a different 5′ and 3′ connector in the core strand. The sequences illustrated in FIG. 13A and 13B are also provided in Table 1 below.

TABLE 1
Sequences of exemplary CASi strands.
mir23 sensor

| | |
|---|---|
| mir23 Sensor 1 | /5Sp9/*mC*+G*mA.+A.mG.mA.+A.mC.mG.+G.mA.+A.mA.mU.+C |
| | .mC.mC.+T.mG.mG.+C.mA*mA*+T*mG*mU*+G*+A*+T*/3CholTE |
| | G/ |
| | (SEQ ID NO: 1) |

Passenger strand

| | |
|---|---|
| HTT V3P1 | +T*+T*mA.+T.mA.mU.mC.mA.fG.mU.fA.fA.fA.mG.mA.mG.mA. |
| | mU.+T *mA*mA |
| | (SEQ ID NO: 2) |

Core strands

| | |
|---|---|
| CASi 1: V3C1 | mU.mC.mC.mG.mU.mU.mC.mU.mU.mC.mG./iSpC3/.mU*fU*mA.m |
| | A.mU.fC.mU.mC.mU.mU.mU.fA.mC.fU.mG.mA.mU.mA.mU.mA.m |
| | A./iSpC3/.mU*mG.mC.mC.mA.mG.mG.mG.mA.mU.mU |
| | (SEQ ID NO: 3, SEQ ID NO: 4, and SEQ ID NO: 5 joined by an |
| | internal C3 spacer ″iSpC3″) |
| CASi 2: V3C2 | mU.mC.mC.mG.mU.mU.mC.mU.mU.mC.mG./iSpC3/.mU*fU*mA.m |
| | A.mU.fC.mU.mC.mU.mU.mU.fA.mC.fU.mG.mA.mU.mA.mU.mA.m |
| | A*mU*mU.mG.mC.mC.mA.mG.mG.mG.mA.mU.mU |
| | (SEQ ID NO: 6 and SEQ ID NO: 7 joined by iSpC3) |
| CASi 3: V3C3 | mU.mC.mC.mG.mU.mU.mC.mU.mU.mC.mG.mA.mU*fU*mA.mA.mU. |
| | fC.mU.mC.mU.mU.mU.fA.mC.fU.mG.mA.mU.mA.mU.mA.mA./iS |
| | pC3/.mU*mG.mC.mC.mA.mG.mG.mG.mA.mU.mU |
| | (SEQ ID NO: 8 and SEQ ID NO: 9 joined by iSpC3) |
| CASi 4: V3C4 | mU.mC.mC.mG.mU.mU.mC.mU.mU.mC.mG.mA.mU* fU*mA.mA.mU. |
| | fC.mU.mC.mU.mU.mU.fA.mC.fU.mG.mA.mU.mA.mU.mA.mA*mU* |
| | mU.mG.mC.mC.mA.mG.mG.mG.mA.mU.mU |
| | (SEQ ID NO: 10) |
| CASi 5: V3C5 | mU.mC.mC.mG.mU.mU.mC.mU.mU.mC.mG.mU*fU*mA.mA.mU.fC. |
| | mU.mC.mU.mU.mU.fA.mC.fU.mG.mA.mU.mA.mU.mA.mA*mU*mU. |
| | mG.mC.mC.mA.mG.mG.mG.mA.mU.mU |
| | (SEQ ID NO: 11) |
| CASi 6: V3C6 | mU.mC.mC.mG.mU.mU.mC.mU.mU.mC.mG.mU*fU*mA.mA.mU.fC. |
| | mU.mC.mU.mU.mU.fA.mC.fU.mG.mA.mU.mA.mU.mA.mA*mU*mG. |
| | mC.mC.mA.mG.mG.mG.mA.mU.mU |
| | (SEQ ID NO: 12) |

/Sp9/ = triethylene glycol spacer
CholTEG = Cholesterol-TEG
/iSpC3/ = internal C3 spacer
* = phosphorothioate backbone
. = phosphodiester backbone
mA, mG, mC, mU = 2′-O-methyl bases
+A, +T, +C, +G = locked nucleic acid (LNA) bases
fA, fU, fC, fG = 2′-fluoro bases
NH2 = primary amine linker.
rA, rU, rC, rG = RNA

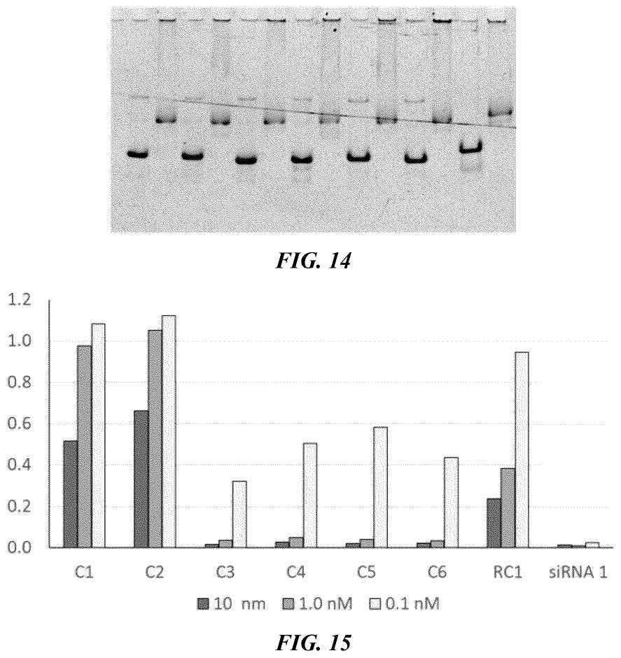

FIG. 14 shows non-denaturing polyacrylamide gel (PAGE) of various nucleic acid complex constructs, indicating all the complexes are assembled as desired. Lanes are as follows (from left to right): P1C1; PI1C1S2; PI1C2; P1C2S2; P1C3; P1C3S2; PI1C4; P1C4S2; P1C5; P1C5S2; P1C6; PI1C6S2; G1RC1; and G1RC1S2. PI indicates the passenger strand 1.

FIG. 15 shows the RNAi activity of two-stranded assemblies each having the same passenger strand v3p1 and a different core strand (C1, C2, C3, C4, C5, and C6) at different concentrations. The sequences of the passenger strand and the core strand are shown in FIGS. 13A and 13B.

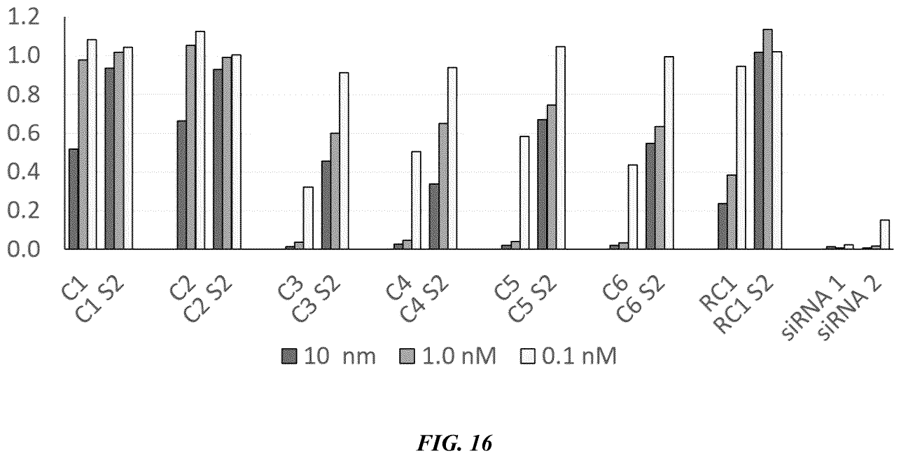

FIG. 16 shows the RNAi activity of three-stranded assemblies each having the same passenger strand v3p1, the same sensor strand (Mir23 sensor 1), and a different core strand (C1, C2, C3, C4, C5, and C6) at three different concentrations. The sequences of the passenger strand, the sensor strand, the core strand are shown in FIGS. 13A and 13B.

These data indicate that assemblies, including two-stranded and three-stranded assembles, with 5′ mA connector and 3′ C3 (3-carbon linker) connector has the highest RNAi activity. Assemblies, including two-stranded and three-stranded assembles, which do not have a 5′ C3 connector (such as C3, C4, C5, C6) have a higher RNAi activity than assemblies having a 5′ C3 connector (C1 and C2). Assemblies that do not have a 5′ connector (C5 and C6) have a lower RNAi activity than assemblies (C3 and C4) having a 5′ connector (such as mA) but not a C3 linker. For the same core strand, the three-stranded assemblies are generally expected to have lower RNAi activity than two-stranded assemblies.

### Example 3

### RNAi Activity of Various RNA Complex Designs

In this example, experiments were carried out to compare the RNAi switching and RNAi activity of Design 1 shown in FIG. 1. and the RNA complex design disclosed herein (e.g., Design 2 shown in FIG. 2). V3C3a and V3C3b are the constructs in the form of Design 2.G1C1S1 is a construct in the form of the Design 1.

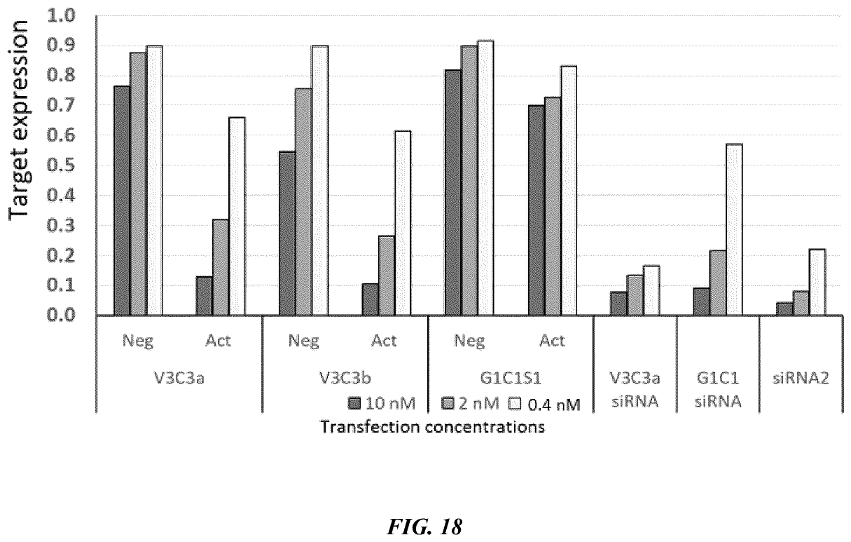

CASi siRNA segment (two-stranded constructs) and three-stranded constructs were assembled by thermally annealing passenger and core strands, or passenger, core and sensor strands in 1x phosphate buffer saline. The CASi siRNA segment (two-stranded constructs) and three-stranded constructs were co-transfected into HCT 116 cells using lipofectamine 2000. The HCT116 cells can express either an RNA biomarker that could activate the CASi sensor (e.g., NPPA gene sequence encoding atrial natriuretic peptide (ANP)) (denoted as “Act” in FIG. 18) or a control nucleic acid strand that could not activate the CASi sensor (denoted as “Neg” in FIG. 18)) using a short RNA transcript driven by a Pol III promoter. The HCT 116 cells also have a dual luciferase vector carrying the calcineurin gene siRNA target sequence. Calcineurin is a calcium and calmodulin dependent serine/threonine protein phosphatase, and has been identified as a key driver of cardiac hypertrophy. ANP has been used as diagnostic markers for cardiac hypertrophy. Therefore, the sensor strand of the three-stranded CASi siRNA constructs is designed to detect ANP mRNA while the siRNA domain (e.g., the passenger strand) is designed to inhibit calcineurin.

After 72 hours, cells were lysed and assayed for knockdown of the target gene (calcineurin) by comparing the luminescence value of Renilla luciferase (carrying the target sequence) to Firefly luciferase.

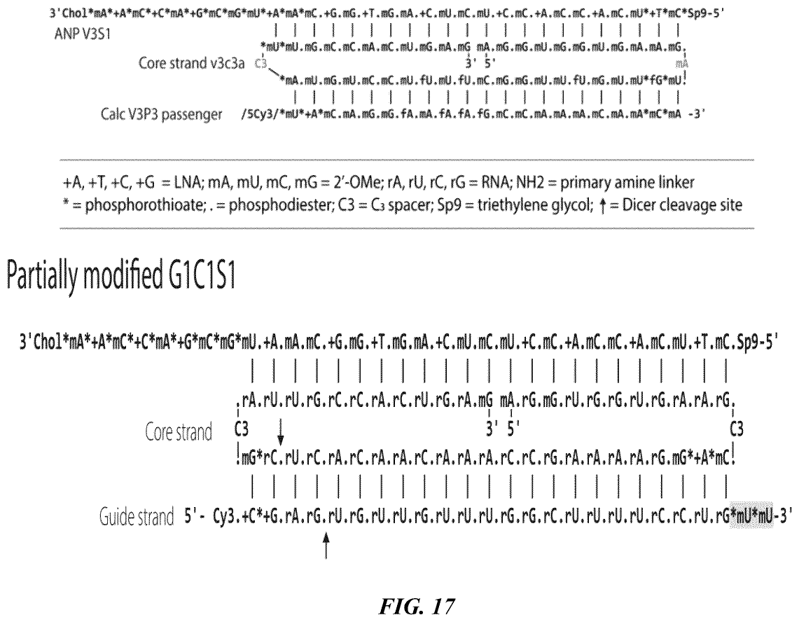

FIG. 17 shows sequence diagrams of a nuclei acid complex including a core strand V3C3a in the form of Design 2 (T2 CASi) shown in FIG. 1 and a nucleic acid complex in the form of Design 1 (Cond-siRNA construct) shown in FIG. 1 (bottom: GIC1S1). The sequences of T2 CASi and Cond-siRNA strands are provided in Table 2.

TABLE 2
Sequences of T2 CASi of Design 2 and Cond-siRNA of Design 1.
Passenger for T2 ANP-calcineurin CASi

| | |
|---|---|
| Calc V3P3 | /5Cy3/*mU*+A*mC.mA.mG.mG.fA.mA.fA.fA.fG.mC.mC.mA.mA.m |
| passenger | A.mC.mA.mA*mC*mA |
| | (SEQ ID NO: 13) |

Sensor strand for both T2 CASi and Cond-siRNA

| | |
|---|---|
| ANP | /5Sp9/.mC.+T.mU.mC.+A.mC.mC.+A.mC.+C.mU.mC.mU.+C.mA.m |
| Sensor 1 | G.+T.mG.+G.mC.mA.+A.mU*mG*mC*+G*mA*+C*mC*+A*mA*/3TEGC |
| | hol/ |
| | (SEQ ID NO: 14) |

Core strand for T2 ANP-calcineurin CASi

| | |
|---|---|
| Rat ANP | mA.mG.mG.mU.mG.mG.mU.mG.mA.mA.mG.mA.mU*fG*mU.mU.mG.fU |
| V3C3a | .mU.mU.mG.mG.mC.fU.mU.fU.mU.mC.mC.mU.mG.mU.mA*/iSpC3/ |
| | *mU*mU.mG.mC.mC.mA.mC.mU.mG.mA.mG |
| | (SEQ ID NO: 15 and SEQ ID NO: 16 joined by iSpC3) |

PPP3CA guide strand for Cond-siRNA

| | |
|---|---|
| Calc G4 | /5Cy3/.+C*+G.rA.rG.rU.rG.rU.rU.rG.rU.mU.rU.mG.mG.mC.r |
| | U.mU.rU.rU.rC.mC.mU.mG*mU*mU |
| | (SEQ ID NO: 17) |

Core strand for Cond-siRNA

| | |
|---|---|
| ANP-Calc | mA.mG.mG.mU.mG.mG.mU.mG.mA.mA.mG./iSpC3/.mC*+A*mG.rG. |
| core | rA.rA.rA.rA.rG.rC.rC.rA.rA.rA.rC.rA.rA.rC.rA.rC.rU.rC |
| strand | *mG./iSpC3/.mA.mU.mU.mG.mC.mC.mA.mU.mU.mG.mA.mG |
| | (SEQ ID NO: 18, SEQ ID NO: 19 and SEQ ID NO: 20 joined by iSpC3) |

/Sp9/ = triethylene glycol spacer
CholTEG = Cholesterol-TEG
/iSpC3/ = internal C3 spacer
* = phosphorothioate backbone
. = phosphodiester backbone
mA, mG, mC, mU = 2′-O-methyl bases
+A, +T, +C, +G = locked nucleic acid (LNA) bases
fA, fU, fC, fG = 2′-fluoro bases
NH2 = primary amine linker.
rA, rU, rC, rG = RNA

FIG. 18 shows the RNAi activity of the modified two-stranded constructs (V3C3a siRNA) and three-stranded constructs (V3C3a and V3C3b) in comparison with the original two-stranded (G1C1 siRNA) and three-stranded constructs (G1C1S1) at three different concentrations.

These data indicate that the modified CASi constructs shows lower RNAi activity in the absence of the RNA biomarker (Neg) and higher RNAi activity in the presence of the RNAi biomarker (Act), thus indicating that the RNAi activity of the modified CASi constructs is switched OFF when the RNA biomarker is absent. The RNAi activity of the modified constructs (V3C3a and V3C3b) was also significantly improved compared to the original design (G1C1S1). The modified CASi siRNA segments (two-stranded assemblies, e.g., V3C3a siRNA)) also show significantly improved RNAi activity compared to the original two-stranded design (G1C1 siRNA).

### Example 4

### Determination of RNAi Activity

This example describes performing RNAi activity of various nucleic acid complex constructs described herein.

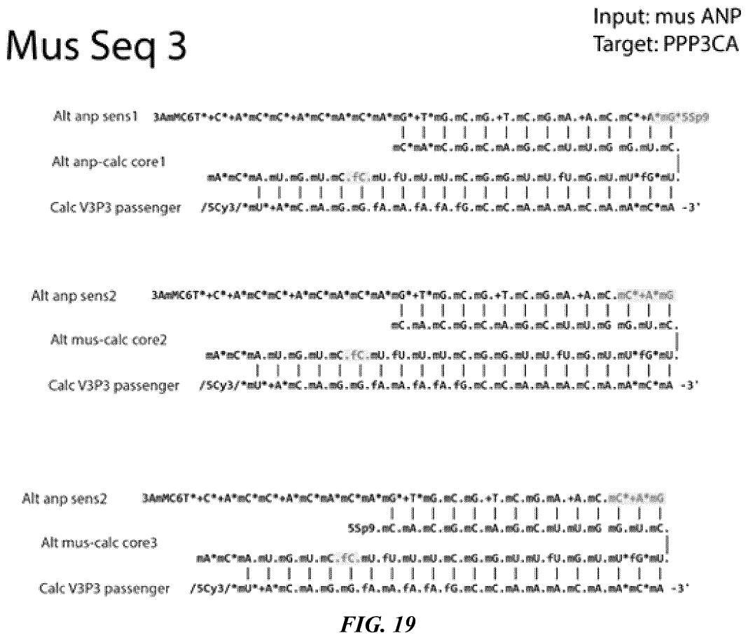

Different variants of the CASi siRNA constructs shown in FIG. 19 can be tested for RNAi activity. The sensor strand of the constructs can be designed to sense an input nucleic acid, such as a NPPA gene sequence encoding atrial natriuretic peptide (ANP). To test the constructs, CASi siRNA constructs can be assembled by thermally annealing the passenger strand, the core strand and the sensor strand in 1× phosphate buffer saline. The RNAi activities of the CASi siRNA constructs can be measured using dual luciferase assays. CASi siRNA constructs can be co-transfected into HCT 116 cells with dual luciferase vectors carrying a calcineurin gene target sequence (PPP3A), using lipofectamine 2000. After 48 hours, cells can be lysed and assayed for knockdown of the target gene by comparing the luminescence value of Renilla luciferase that carries the target sequence to Firefly luciferase that can be used as a reference control. Examples of methods and procedures of assembling CASi siRNA constructs, cell transfection, and dual luciferase assays are described in, for example, international application WO/2020/033938, the content of which is incorporated herein by reference in its entirety. It is expected that the RNA complexes described herein have RNAi activities.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A nucleic acid complex, comprising:
a first nucleic acid strand comprising 20-70 linked nucleosides;
a second nucleic acid strand binding to a central region of the first nucleic acid strand to form a first nucleic acid duplex; and
a third nucleic acid strand binding to a 5′ region and a 3′ region of the first nucleic acid strand to form a second nucleic acid duplex, wherein the third nucleic acid strand comprises an overhang, wherein the overhang is not complementary to the first nucleic acid strand and is capable of binding to an input nucleic acid strand to cause the displacement of the third nucleic acid strand from the first nucleic acid strand,
wherein the second nucleic acid duplex comprises at least one wobble base pair.

2. The nucleic acid complex of claim 1, wherein the at least one wobble base pair is selected from a group consisting of: a guanine-uracil (G-U) wobble base pair, a hypoxanthine-uracil (I-U) wobble base pair, a hypoxanthine-adenine (I-A) wobble base pair, a hypoxanthine-cytosine (I-C) wobble base pair, or a combination thereof, wherein the at least one wobble base pair is to decrease the melting temperature of the second nucleic acid duplex.

3. (canceled)

4. (canceled)

5. The nucleic acid complex of claim 1, wherein the 5′ region, the 3′ region, or both, of the first nucleic acid strand comprise one or more universal base; wherein
the universal base is selected from the group consisting of: hypoxanthine and derivatives thereof, inosine and derivatives thereof, azole carboxamide and derivatives thereof, nitroazole and derivatives thereof, phenyl C-ribonucleoside and derivatives thereof, naphthyl C-ribonucleoside and derivatives thereof, or a combination thereof; or wherein the one or more universal base comprises inosine.

6. (canceled)

7. (canceled)

8. The nucleic acid complex of claim 1, wherein the central region of the first nucleic acid strand comprises a sequence complementary to a target RNA, wherein the sequence complementary to the target RNA is 10-21 nucleotides in length; and wherein the second nucleic acid strand binds to 10-21 linked nucleotides in the central region of the first nucleic acid strand to form the first nucleic acid duplex; and
wherein the first nucleic acid duplex does not comprise a Dicer cleavage site or wherein the nucleic acid complex does not comprise a Dicer cleavage site.

9. (canceled)

10. (canceled)

11. (canceled)

12. The nucleic acid complex of claim 1, wherein the central region of the first nucleic acid strand is linked to the 5′ region of the first nucleic acid strand via a 5′ connector, the central region of the first nucleic acid strand is linked to the 3′ region of the first nucleic acid strand via a 3′ connector, or both; and
wherein the 5′ connector, the 3′ connector, or both comprise a C₃ 3-carbon linker, a nucleotide, a modified nucleotide, or a exonuclease cleavage-resistant moiety, or a combination thereof; wherein the modified nucleotide is a 2′-O-methyl nucleotide or a 2′-F nucleotide.

13. (canceled)

14. (canceled)

15. (canceled)

16. (canceled)

17. (canceled)

18. (canceled)

19. The nucleic acid complex of claim 1, wherein the second nucleic strand is fully complementary to the central region of the first nucleic acid strand, thereby forming blunt ends at the 5′ and 3′ termini of the second nucleic acid strand in the first nucleic acid duplex.

20. (canceled)

21. (canceled)

22. (canceled)

23. The nucleic acid complex of claim 1, wherein the 5′ terminus of the central region of the first nucleic acid strand, the 3′ terminus of the central region of the first nucleic acid strand, or both, comprises at least one phosphorothioate internucleoside linkage; or
wherein the central region of the first nucleic acid strand does not comprise phosphorothioate internucleoside linkages except for the internucleoside linkage(s) between two or three nucleosides at the 5′ terminus, 3′ terminus, or both, of the central region.

24. (canceled)

25. (canceled)

26. (canceled)

27. The nucleic acid complex of claim 1, wherein at least 80%, at least 85%, at least 90%, or at least 95% of the nucleosides of one or more of the first nucleic acid strand, the second nucleic strand and the third nucleic strand are chemically modified; or wherein at least 80%, at least 85%, at least 90%, at least 95%, or all of the nucleosides of the nucleic acid complex are chemically modified.

28. (canceled)

29. (canceled)

30. (canceled)

31. (canceled)

32. (canceled)

33. (canceled)

34. (canceled)

35. (canceled)

36. (canceled)

37. The nucleic acid complex of claim 1, wherein the internucleoside linkages between (1) the one to three nucleotides adjacent to the 3′ of the 5′ connector, and/or (2) the one or two nucleotides adjacent to the 5′ of the 3′ connector, and/or (3) the one to three nucleotides adjacent to the 3′ of the 3′ connector, are phosphorothioate internucleoside linkages.

38. (canceled)

39. (canceled)

40. The nucleic acid complex of claim 1, wherein the overhang of the third nucleic acid strand is capable of binding to the input nucleic acid strand to form a toehold, thereby causing the displacement of the third nucleic acid strand from the first nucleic acid strand;
wherein the overhang of the third nucleic acid strand is 5 to 20 nucleosides in length;
wherein all internucleoside linkages of the overhang of the third nucleic acid strand are phosphorothioate internucleoside linkages; and
wherein the 5′ terminus, the 3′ terminus, or both of the third nucleic acid strand comprises a terminal moiety; wherein the terminal moiety comprises a ligand, a fluorophore, a exonuclease, a fatty acid, a Cy3, an inverted dT attached to a tri-ethylene glycol, or a combination thereof.

41. (canceled)

42. (canceled)

43. (canceled)

44. A nucleic acid complex, comprising:
a first nucleic acid strand comprising 20-60 linked nucleosides;
a second nucleic acid strand binding to a first region of the first nucleic acid strand to form a first nucleic acid duplex; and
a third nucleic acid strand binding to a second region of the first nucleic acid strand to form a second nucleic acid duplex, wherein the third nucleic acid strand comprises a overhang, wherein the overhang is not complementary to the first nucleic acid strand and is capable of binding to an input nucleic acid strand to cause the displacement of the third nucleic acid strand from the first nucleic acid strand,
wherein
the first region of the first nucleic acid strand is 3′ of the second region of the first nucleic acid strand,
the third nucleic acid strand does not bind to any region of the first nucleic acid strand that is 3′ of the first region of the first nucleic acid strand, and
the second nucleic acid duplex comprises at least one wobble base pair.

45. The nucleic acid complex of claim 44, wherein the at least one wobble base pair is selected from a group consisting of: a guanine-uracil (G-U) wobble base pair, a hypoxanthine-uracil (I-U) wobble base pair, a hypoxanthine-adenine (I-A) wobble base pair, a hypoxanthine-cytosine (I-C) wobble base pair, or a combination thereof, wherein the at least one wobble base pair is to decrease the melting temperature of the second nucleic acid duplex.

46. (canceled)

47. The nucleic acid complex of claim 44, wherein the second region of the first nucleic acid strand comprises one or more universal base and/or wherein a portion of the third nucleic acid strand that binds to the second region of the first nucleic acid strand comprises one or more universal base;
wherein the universal base is selected from the group consisting of: hypoxanthine and derivatives thereof, inosine and derivatives thereof, azole carboxamide and derivatives thereof, nitroazole and derivatives thereof, phenyl C-ribonucleoside and derivatives thereof, naphthyl C-ribonucleoside and derivatives thereof, or a combination thereof, or
wherein the one or more universal base comprises inosine.

48. (canceled)

49. (canceled)

50. (canceled)

51. The nucleic acid complex of claim 44, wherein: the first region of the first nucleic acid strand comprises a sequence complementary to a target RNA, wherein the sequence complementary to the target RNA is 10-21 nucleotides in length;
wherein the second nucleic acid strand binds to 10-21 linked nucleotides in the first region of the first nucleic acid strand to form the first nucleic acid duplex,
wherein the third nucleic acid strand binds to 10-30 linked nucleotides in the second region of the first nucleic acid strand to form the second nucleic acid duplex; and
wherein the first nucleic acid duplex does not comprise a Dicer cleavage site or wherein the nucleic acid complex does not comprise a Dicer cleavage site.

52. (canceled)

53. (canceled)

54. (canceled)

55. (canceled)

56. (canceled)

57. The nucleic acid complex of claim 44, wherein the first region of the first nucleic acid strand is linked to the second region of the first nucleic acid strand via a linker, wherein the linker comprises a C₃ 3-carbon linker, a nucleotide, a modified nucleotide, or a exonuclease cleavage-resistant moiety, or a combination thereof, wherein the modified nucleotide is a 2′-O-methyl nucleotide or a 2′-F nucleotide.

58. (canceled)

59. (canceled)

60. (canceled)

61. (canceled)

62. The nucleic acid complex of claim 44, wherein the 5′ terminus of the second nucleic acid strand comprises a blocking moiety; wherein the blocking moiety comprises, or is, a fluorophore, an inverted-dT, a tri-ethylene-glycol, a fatty acid, a Cy3, or a combination thereof.

63. The nucleic acid complex of claim 44, wherein the first nucleic acid strand comprises a 3′ overhang that is one, two, or three nucleotides in length in the first nucleic acid duplex; wherein the 3′ overhang of the first nucleic acid comprises one or more phosphorothioate internucleoside linkages; and
wherein the first region of the first nucleic acid strand does not comprise phosphorothioate internucleoside linkages except for the internucleoside linkage(s) between the last two or three nucleosides at the 5′ terminus, 3′ terminus, or both; and wherein the second region of the first nucleic acid strand does not comprise phosphorothioate internucleoside linkages.

64. (canceled)

65. (canceled)

66. (canceled)

67. (canceled)

68. (canceled)

69. (canceled)

70. (canceled)

71. The nucleic acid complex of claim 44, wherein the second nucleic strand is fully complementary to the first region of the first nucleic acid strand, thereby forming no overhang at the 5′ and 3′ termini of the second nucleic acid strand in the first nucleic acid duplex, wherein the second nucleic acid strand comprises one or more phosphorothioate internucleoside linkages or wherein the second nucleic acid strand does not comprise phosphorothioate internucleoside linkages except for the internucleoside linkage(s) between the last two to three nucleosides at the 5′ terminus and the last two to three nucleosides at 3′ terminus; and
wherein the last two, three or four nucleosides at the 5′ terminus of the third nucleic acid strand are phosphorothioate internucleoside linkages.

72. (canceled)

73. (canceled)

74. (canceled)

75. (canceled)

76. (canceled)

77. (canceled)

78. (canceled)

79. (canceled)

80. (canceled)

81. The nucleic acid complex of claim 44, wherein at least 50%, at least 60%, at least 70%, at least 80%, at least 85%, at least 90%, at least 95%, or all of the nucleosides of one or more of the first nucleic acid strand, the second nucleic strand and the third nucleic strand are chemically modified; or wherein at least 50%, at least 60%, at least 70%, at least 80%, at least 85%, at least 90%, at least 95%, or all of the nucleosides of the nucleic acid complex are chemically modified.

82. (canceled)

83. (canceled)

84. (canceled)

85. (canceled)

86. (canceled)

87. (canceled)

88. (canceled)

89. (canceled)

90. The nucleic acid complex of claim 44, wherein the overhang of the third nucleic acid strand is capable of binding to the input nucleic acid strand to form a toehold, thereby causing the displacement of the third nucleic acid strand from the first nucleic acid strand; wherein the overhang of the third nucleic acid strand is 5 to 20 nucleosides in length and wherein all internucleoside linkages of the overhang of the third nucleic acid strand are phosphorothioate internucleoside linkages; and
wherein the 5′ terminus, the 3′ terminus, or both of the third nucleic acid strand comprises a terminal moiety; and optionally the terminal moiety comprises a ligand, a fluorophore, a exonuclease, a fatty acid, a Cy3, an inverted dT attached to a tri-ethylene glycol, or a combination thereof.

91. (canceled)

92. (canceled)

93. (canceled)
